SEQUENCE LISTING (136 sequences)

SEQ ID NO: 1 - DNA, 105 bp, Homo sapiens
  misc_feature (26)..(75): Exon 5
agggatttgctttgttttattttagtcctgttgttctacaatgtacacatgtaacaccacaaagagataagtcaggtatgattaaaaacaatgctttttattctt

SEQ ID NO: 2 - DNA, 218 bp, Homo sapiens
  misc_feature (29)..(210): Exon 15
tttttgctaagtatttattctttgatagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacaggtatgtgt

SEQ ID NO: 3 - DNA, 258 bp, Homo sapiens
  misc_feature (34)..(221): Exon 16
tttttcttttttgtgtgtgtttattttgtgtagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttataggtactctatgcaaaaagattgtgtgttaacttttatg

SEQ ID NO: 4 - DNA, 10485 bp, Homo sapiens
  CDS (229)..(10482): BRCA2 (OMI1)
ggtggcgcgagcttctgaaactaggcggcagaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcacatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacatcttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctgttgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtctcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagcagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaa

SEQ ID NO: 5 - protein, 3418 aa, Homo sapiens [sequence omitted]

SEQ ID NO: 6 - DNA, 10485 bp, Homo sapiens
  CDS (229)..(10482): BRCA2 (OMI2)
ggtggcgcgagcttctgaaactaggcggcagaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcacatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacatcttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaarccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgtcgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctgttgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtctcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagcagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaa

SEQ ID NO: 7 - protein, 3418 aa, Homo sapiens [sequence omitted]

SEQ ID NO: 8 - DNA, 10485 bp, Homo sapiens
  CDS (229)..(10482): BRCA2 (OMI3)
ggtggcgcgagcttctgaaactaggcggcagaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccacatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcaaatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacatcttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacactcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatggacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaagccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctgttgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtctcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcgcattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagcagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaa

SEQ ID NO: 9 - protein, 3418 aa, Homo sapiens
MPIGSKERPTFFEIFKTRCNKADLGPISLNWFEELSSEAPPYNSEPAEESEHKNNNYEPNLFKTPQRKPSYNQLASTPIIFKEQGLTLPLYQSPVKELDKFKLDLGRNVPNSRHKSLRTVKTKMDQADDVSCPLLNSCLSESPVVLQCTHVTPQRDKSVVCGSLFHTPKFVKGRQTPKHISESLGAEVDPDMSWSSSLATPPTLSSTVLIVRNEEASETVFPHDTTANVKSYFSNHDESLKKNDRFIASVTDSENTNQREAASHGFGKTSGNSFKVNSCKDHIGKSMPHVLEDEVYETVVDTSEEDSFSLCFSKCRTKNLQKVRTSKTRKKIFHEANADECEKSKNQVKEKYSFVSEVEPNDTDPLDSNVANQKPFESGSDKISKEVVPSLACEWSQLTLSGLNGAQMEKIPLLHISSCDQNISEKDLLDTENKRKKDFLTSENSLPRISSLPKSEKPLNEETVVNKRDEEQHLESHTDCILAVKQAISGTSPVASSFQGIKKSIFRIRESPKETFNASFSGHMTDPNFKKETEASESGLEIHTVCSQKEDSLCPNLIDNGSWPATTTQNSVALKNAGLISTLKKKTNKFIYAIHDETSYKGKKIPKDQKSELINCSAQFEANAFEAPLTFANADSGLLHSSVKRSCSQNDSEEPTLSLTSSFGTILRKCSRNETCSNNTVISQDLDYKEAKCNKEKLQLFITPEADSLSCLQEGQCENDPKSKKVSDIKEEVLAAACHPVQHSKVEYSDTDFQSQKSLLYDHENASTLILTPTSKDVLSNLVMISRGKESYKMSDKLKGNNYESDVELTKNIPMEKNQDVCALNENYKNVELLPPEKYMRVASPSRKVQFNQNTNLRVIQKNQEETTSISKITVNPDSEELFSDNENNFVFQVANERNNLALGNTKELHETDLTCVNEPIFKNSTMVLYGDTGDKQATQVSIKKDLVYVLAEENKNSVKQHIKMTLGQDLKSDISLNIDKIPEKNNDYMDKWAGLLGPISNHSFGGSFRTASNKEIKLSEHNIKKSKMFFKDIEEQYPTSLACVEIVNTLALDNQKKLSKPQSINTVSAHLQSSVVVSDCKNSHITPQMLFSKQDFNSNHNLTPSQKAEITELSTILEESGSQFEFTQFRKPSYILQKSTFEVPENQMTILKTTSEECRDADLHVIMNAPSIGQVDSSKQFEGTVEIKRKFAGLLKNDCNKSASGYLTDENEVGFRGFYSAHGTKLNVSTEALQKAVKLFSDIENISEETSAEVHPISLSSSKCHDSVVSMFKIENHNDKTVSEKNNKCQLILQNNIEMTTGTFVEEITENYKRNTENEDNKYTAASRNSHNLEFDGSDSSKNDTVCIHKDETDLLFTDQHNICLKLSGQFMKEGNTQIKEDLSDLTFLEVAKAQEACHGNTSNKEQLTATKTEQNIKDFETSDTFFQTASGKNISVAKESFNKIVNFFDQKPEELHNFSLNSELHSDIRKNKMDILSYEETDIVKHKILKESVPVGTGNQLVTFQGQPERDEKIKEPTLLGFHTASGKKVKIAKESLDKVKNLFDEKEQGTSEITSFSHQWAKTLKYREACKDLELACETIEITAAPKCKEMQNSLNNDKNLVSIETVVPPKLLSDNLCRQTENLKTSKSIFLKVKVHENVEKETAKSPATCYTNQSPYSVIENSALAFYTSCSRKTSVSQTSLLEAKKWLREGIFDGQPERINTADYVGNYLYENNSNSTIAENDKNHLSEKQDTYLSNSSMSNSYSYHSDEVYNDSGYLSKNKLDSGIEPVLKNVEDQKNTSFSKVISNVKDANAYPQTVNEDICVEELVTSSSPCKNKNAAIKLSISNSNNFEVGPPAFRIASGKIVCVSHETIKKVKDIFTDSFSKVIKENNENKSKICQTKIMAGCYEALDDSEDILHNSLDNDECSTHSHKVFADIQSEEILQHNQNMSGLEKVSKISPCDVSLETSDICKCSIGKLHKSVSSANTCGIFSTASGKSVQVSDASLQNARQVFSEIEDSTKQVFSKVLFKSNEHSDQLTREENTAIRTPEHLISQKGFSYNVVNSSAFSGFSTASGKQVSILESSLHKVKGVLEEFDLIRTEHSLHYSPTSRQNVSKILPRVDKRNPEHCVNSEMEKTCSKEFKLSNNLNVEGGSSENNHSIKVSPYLSQFQQDKQQLVLGTKVSLVENIHVLGKEQASPKNVKMEIGKTETFSDVPVKTNIEVCSTYSKDSENYFETEAVEIAKAFMEDDELTDSKLPSHATHSLFTCPENEEMVLSNSRIGKRRGEPLILVGEPSIKRNLLNEFDRIIENQEKSLKASKSTPDGTIKDRRLFMHHVSLEPITCVPFRTTKERQEIQNPNFTAPGQEFLSKSHLYEHLTLEKSSSNLAVSGHPFYQVSATRNEKMRHLITTGRPTKVFVPPFKTKSHFHRVEQCVRNINLEENRQKQNIDGHGSDDSKNKINDNEIHQFNKNNSNQAAAVTFTKCEEEPLDLITSLQNARDIQDMRIKKKQRQRVFPQPGSLYLAKTSTLPRISLKAAVGGQVPSACSHKQLYTYGVSKHCIKINSKNAESFQFHTEDYFGKESLWTGKGIQLADGGWLIPSNDGKAGKEEFYRALCDTPGVDPKLISRIWVYNHYRWIIWKLAAMECAFPKEFANRCLSPERVLLQLKYRYDTEIDRSRRSAIKKIMERDDTAAKTLVLCVSDIISLSANISETSSNKTSSADTQKVAIIELTDGWYAVKAQLDPPLLAVLKNGRLTVGQKIILHGAELVGSPDACTPLEAPESLMLKISANSTRPARWYTKLGFFPDPRPFPLPLSSLFSDGGNVGCVDVIIQRAYPIQWMEKTSSGLYIFRNEREEEKEAAKYVEAQQKRLEALFTKIQEEFEEHEENTTKPYLPSRALTRQQVRALQDGAELYEAVKNAADPAYLEGYFSEEQLRALNNHRQMLNDKKQAQIQLEIRKAMESAEQKEQGLSRDVTTVWKLRIVSYSKKEKDSVILSIWRPSSDLYSLLTEGKRYRIYHLATSKSKSKSERANIQLAATKKTQYQQLPVSDEILFQIYQPREPLHFSKFLDPDFQPSCSEVDLIGFVVSVVKKTGLAPFVYLSDECYNLLAIKFWIDLNEDIIKPHMLIAASNLQWRPESKSGLLTLFAGDFSVFSASPKEGHFQETFNKMKNTVENIDILCNEAENKLMHILHANDPKWSTPTKDCTSGPYTAQIIPGTGNKLLMSSPNCEIYYQSPLSLCMAKRKSVSTPVSAQMTSKSCKGEKEIDDQKNCKKRRALDFLSRLPLPPPVSPICTFVSPAAQKAFQPPRSCGTKYETPIKKKELNSPQMTPFKKFNEISLLESNSIADEELALINTQALLSGSTGEKQFISVSESTRTAPTSSEDYLRLKRRCTTSLIKEQESSQASTEECEKNKQDTITTKKYI

SEQ ID NO: 10 - DNA, 10485 bp, Homo sapiens
  CDS (229)..(10482): BRCA2 (OMI4)
ggtggcgcgagcttctgaaactaggcggcagaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcacatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacatcttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaagccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctgttgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtctcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcgcattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagcagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaa

SEQ ID NO: 11 - protein, 3418 aa, Homo sapiens [sequence omitted]

SEQ ID NO: 12 - DNA, 10485 bp, Homo sapiens
  CDS (229)..(10482): BRCA2 (OMI5)
ggtggcgcgagcttctgaaactaggcggcagaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccacatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcacatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcggagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacatcttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacactcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaaatagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatggacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctgttgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtctcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagcagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaagaccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaa

SEQ ID NO: 13 - protein, 3418 aa, Homo sapiens
MPIGSKERPTFFEIFKTRCNKADLGPISLNWFEELSSEAPPYNSEPAEESEHKNNNYEPNLFKTPQRKPSYNQLASTPIIFKEQGLTLPLYQSPVKELDKFKLDLGRNVPNSRHKSLRTVKTKMDQADDVSCPLLNSCLSESPVVLQCTHVTPQRDKSVVCGSLFHTPKFVKGRQTPKHISESLGAEVDPDMSWSSSLATPPTLSSTVLIVRNEEASETVFPHDTTANVKSYFSNHDESLKKNDRFIASVTDSENTNQREAASHGFGKTSGNSFKVNSCKDHIGKSMPHVLEDEVYETVVDTSEEDSFSLCFSKCRTKNLQKVRTSKTRKKIFHEANADECEKSKNQVKEKYSFVSEVEPNDTDPLDSNVAHQKPFESGSDKISKEVVPSLACEWSQLTLSGLNGAQMEKIPLLHISSCDQNISEKDLLDTENKRKKDFLTSENSLPRISSLPKSEKPLNEETVVNKRDEEQHLESHTDCILAVKQAISGTSPVASSFQGIKKSIFRIRESPKETFNASFSGHMTDPNFKKETEASESGLEIHTVCSQKEDSLCPNLIDNGSWPATTTQNSVALKNAGLISTLKKKTNKFIYAIHDETSYKGKKIPKDQKSELINCSAQFEANAFEAPLTFANADSGLLHSSVKRSCSQNDSEEPTLSLTSSFGTILRKCSRNETCSNNTVISQDLDYKEAKCNKEKLQLFITPEADSLSCLQEGQCENDPKSKKVSDIKEEVLAAACHPVQHSKVEYSDTDFQSQKSLLYDHENASTLILTPTSKDVLSNLVMISRGKESYKMSDKLKGNNYESDVELTKNIPMEKNQDVCALNENYKNVELLPPEKYMRVASPSRKVQFNQNTNLRVIQKNQEETTSISKITVNPDSEELFSDNENNFVFQIANERNNLALGNTKELHETDLTCVNEPIFKNSTMVLYGDTGDKQATQVSIKKDLVYVLAEENKNSVKQHIKMTLGQDLKSDISLNIDKIPEKNNDYMDKWAGLLGPISNHSFGGSFRTASNKEIKLSEHNIKKSKMFFKDIEEQYPTSLACVEIVNTLALDNQKKLSKPQSINTVSAHLQSSVVVSDCKNSHITPQMLFSKQDFNSNHNLTPSQKAEITELSTILEESGSQFEFTQFRKPSYILQKSTFEVPENQMTILKTTSEECRDADLHVIMNAPSIGQVDSSKQFEGTVEIKRKFAGLLKNDCNKSASGYLTDENEVGFRGFYSAHGTKLNVSTEALQKAVKLFSDIENISEETSAEVHPISLSSSKCHDSVVSMFKIENHNDKTVSEKNNKCQLILQNNIEMTTGTFVEEITENYKRNTENEDNKYTAASRNSHNLEFDGSDSSKNDTVCIHKDETDLLFTDQHNICLKLSGQFMKEGNTQIKEDLSDLTFLEVAKAQEACHGNTSNKEQLTATKTEQNIKDFETSDTFFQTASGKNISVAKESFNKIVNFFDQKPEELHNFSLNSELHSDIRKNKMDILSYEETDIVKHKILKESVPVGTGNQLVTFQGQPERDEKIKEPTLLGFHTASGKKVKIAKESLDKVKNLFDEKEQGTSEITSFSHQWAKTLKYREACKDLELACETIEITAAPKCKEMQNSLNNDKNLVSIETVVPPKLLSDNLCRQTENLKTSKSIFLKVKVHENVEKETAKSPATCYTNQSPYSVIENSALAFYTSCSRKTSVSQTSLLEAKKWLREGIFDGQPERINTADYVGNYLYENNSNSTIAENDKNHLSEKQDTYLSNSSMSNSYSYHSDEVYNDSGYLSKNKLDSGIEPVLKNVEDQKNTSFSKVISNVKDANAYPQTVNEDICVEELVTSSSPCKNKNAAIKLSISNSNNFEVGPPAFRIASGKIVCVSHETIKKVKDIFTDSFSKVIKENNENKSKICQTKIMAGCYEALDDSEDILHNSLDNDECSTHSHKVFADIQSEEILQHNQNMSGLEKVSKISPCDVSLETSDICKCSIGKLHKSVSSANTCGIFSTASGKSVQVSDASLQNARQVFSEIEDSTKQVFSKVLFKSNEHSDQLTREENTAIRTPEHLISQKGFSYNVVNSSAFSGFSTASGKQVSILESSLHKVKGVLEEFDLIRTEHSLHYSPTSRQNVSKILPRVDKRNPEHCVNSEMEKTCSKEFKLSNNLNVEGGSSENNHSIKVSPYLSQFQQDKQQLVLGTKVSLVENIHVLGKEQASPKNVKMEIGKTETFSDVPVKTNIEVCSTYSKDSENYFETEAVEIAKAFMEDDELTDSKLPSHATHSLFTCPENEEMVLSNSRIGKRRGEPLILVGEPSIKRNLLNEFDRIIENQEKSLKASKSTPDGTIKDRRLFMHHVSLEPITCVPFRTTKERQEIQNPNFTAPGQEFLSKSHLYEHLTLEKSSSNLAVSGHPFYQVSATRNEKMRHLITTGRPTKVFVPPFKTKSHFHRVEQCVRNINLEENRQKQNIDGHGSDDSKNKINDNEIHQFNKNNSNQAAAVTFTKCEEEPLDLITSLQNARDIQDMRIKKKQRQRVFPQPGSLYLAKTSTLPRISLKAAVGGQVPSACSHKQLYTYGVSKHCIKINSKNAESFQFHTEDYFGKESLWTGKGIQLADGGWLIPSNDGKAGKEEFYRALCDTPGVDPKLISRIWVYNHYRWIIWKLAAMECAFPKEFANRCLSPERVLLQLKYRYDTEIDRSRRSAIKKIMERDDTAAKTLVLCVSDIISLSANISETSSNKTSSADTQKVAIIELTDGWYAVKAQLDPPLLAVLKNGRLTVGQKIILHGAELVGSPDACTPLEAPESLMLKISANSTRPARWYTKLGFFPDPRPFPLPLSSLFSDGGNVGCVDVIIQRAYPIQWMEKTSSGLYIFRNEREEEKEAAKYVEAQQKRLEALFTKIQEEFEEHEENTTKPYLPSRALTRQQVRALQDGAELYEAVKNAADPAYLEGYFSEEQLRALNNHRQMLNDKKQAQIQLEIRKTMESAEQKEQGLSRDVTTVWKLRIVSYSKKEKDSVILSIWRPSSDLYSLLTEGKRYRIYHLATSKSKSKSERANIQLAATKKTQYQQLPVSDEILFQIYQPREPLHFSKFLDPDFQPSCSEVDLIGFVVSVVKKTGLAPFVYLSDECYNLLAIKFWIDLNEDIIKPHMLIAASNLQWRPESKSGLLTLFAGDFSVFSASPKEGHFQETFNKMKNTVENIDILCNEAENKLMHILHANDPKWSTPTKDCTSGPYTAQIIPGTGNKLLMSSPNCEIYYQSPLSLCMAKRKSVSTPVSAQMTSKSCKGEKEIDDQKNCKKRRALDFLSRLPLPPPVSPICTFVSPAAQKAFQPPRSCGTKYETPIKKKELNSPQMTPFKKFNEISLLESNSIADEELALINTQALLSGSTGEKQFISVSESTRTAPTSSEDYLRLKRRCTTSLIKEQESSQASTEECEKNKQDTITTKKYI

SEQ ID NO: 14 - DNA, 20 bp, Artificial sequence
  BRCA2 Primer Sequence
tgagttttacctcagtcaca

SEQ ID NO: 15 - DNA, 41 bp, Artificial sequence
  BRCA2 Primer Sequence
caggaaacagctatgaccctgtgacgtactgggtttttagc

SEQ ID NO: 16 - DNA, 24 bp, Artificial sequence
  3FII Primer
gatctttaactgttctgggtcaca

SEQ ID NO: 17 - DNA, 22 bp, Artificial sequence
  3RII primer
cccagcatgacacaattaatga

SEQ ID NO: 18 - DNA, 44 bp, Artificial sequence
  4F/M 13F primer
tgtaaaacgacggccagtagaatgcaaatttataatccagagta

SEQ ID NO: 19 - DNA, 22 bp, Artificial sequence
  4R-1A primer
atcagattcatctttatagaac

SEQ ID NO: 20 - DNA, 40 bp, Artificial sequence
  5+6F/M13F primer
tgtaaaacgacggccagttgtgttggcattttaaacatca

SEQ ID NO: 21 - DNA, 38 bp, Artificial sequence
  BRCA2 Primer Sequence
caggaaacagctatgacccagggcaaaggtataacgct

SEQ ID NO: 22 - DNA, 38 bp, Artificial sequence
  BRCA2 Primer Sequence
tgtaaaacgacggccagttaagtgaaataaagagtgaa

SEQ ID NO: 23 - DNA, 36 bp, Artificial sequence
  BRCA2 Primer Sequence
caggaaacagctatgaccagaagtattagagatgac

SEQ ID NO: 24 - DNA, 40 bp, Artificial sequence
  BRCA2 Primer Sequence
tgtaaaacgacggccagtgccatatcttaccaccttgtga

SEQ ID NO: 25 - DNA, 22 bp, Artificial sequence
  BRCA2 Primer Sequence
ttgcattctagtgataatatac

SEQ ID NO: 26 - DNA, 19 bp, Artificial sequence
  BRCA2 Primer Sequence
aattgttagcaatttcaac

SEQ ID NO: 27 - DNA, 40 bp, Artificial sequence
  BRCA2 Primer Sequence
tgtaaaacgacggccagttggacctaggttgattgcagat

SEQ ID NO: 28 - DNA, 40 bp, Artificial sequence
  BRCA2 Primer Sequence
caggaaacagctatgacctaaactgagatcacgggtgaca

SEQ ID NO: 29 - DNA, 24 bp, Artificial sequence
  BRCA2 Primer Sequence
gaataatataaattatatggctta

SEQ ID NO: 30 - DNA, 37 bp, Artificial sequence
  BRCA2 Primer Sequence
caggaaacagctatgacccctagtcttgctagttctt

SEQ ID NO: 31 - DNA, 42 bp, Artificial sequence
  BRCA2 Primer Sequence
tgtaaaacgacggccagtarctgaagtggaaccaaatgatac

SEQ ID NO: 32 - DNA, 44 bp, Artificial sequence
  BRCA2 Primer Sequence
caggaaacagctatgaccacgtggcaaagaattctctgaagtaa

SEQ ID NO: 33 - DNA, 40 bp, Artificial sequence
  BRCA2 Primer Sequence
tgtaaaacgacggccagtcagcatcttgaatctcatacag

SEQ ID NO: 34 - DNA, 19 bp, Artificial sequence
  BRCA2 Primer Sequence
agacagaggtacctgaatc

SEQ ID NO: 35 - DNA, 40 bp, Artificial sequence
  BRCA2 Primer Sequence
tgtaaaacgacggccagttggtactttaattttgtcactt

SEQ ID NO: 36 - DNA, 37 bp, Artificial sequence
  BRCA2 Primer Sequence
caggaaacagctatgacctgcaggcatgacagagaat

SEQ ID NO: 37 - DNA, 22 bp, Artificial sequence
  BRCA2 Primer Sequence
aagaagcaaaatgtaataagga

SEQ ID NO: 38 - DNA, 22 bp, Artificial sequence
  BRCA2 Primer Sequence
catttaaagcacatacatcttg

SEQ ID NO: 39 - DNA, 21 bp, Artificial sequence
  BRCA2 Primer Sequence
tctagaggcaaagaatcatac

SEQ ID NO: 40 - DNA, 22 bp, Artificial sequence
  BRCA2 Primer Sequence
caagattattcctttcattagc

SEQ ID NO: 41 - DNA, 22 bp, Artificial sequence
  BRCA2 Primer Sequence
aaccaaaacacaaatctaagag

SEQ ID NO: 42 - DNA, 23 bp, Artificial sequence
  BRCA2 Primer Sequence
gtcatttttatatgctgctttac

SEQ ID NO: 43 - DNA, 21 bp, Artificial sequence
  BRCA2 Primer Sequence
ggttttatatggagacacagg

SEQ ID NO: 44 - DNA, 23 bp, Artificial sequence
  BRCA2 Primer Sequence
gtatttacaatttcaacacaagc

SEQ ID NO: 45 - DNA, 20 bp, Artificial sequence
  BRCA2 Primer Sequence
atcacagttttggaggtagc

SEQ ID NO: 46 - DNA, 21 bp, Artificial sequence
  BRCA2 Primer Sequence
ctgacttcctgattcttctaa

SEQ ID NO: 47 - DNA, 21 bp, Artificial sequence
  BRCA2 Primer Sequence
ctcagatgttattttccaagc

SEQ ID NO: 48 - DNA, 21 bp, Artificial sequence
  BRCA2 Primer Sequence
ctgttaaataaccagaagcac

SEQ ID NO: 49 - DNA, 18 bp, Artificial sequence
  BRCA2 Primer Sequence
aggtagacagcagcaagc

SEQ ID NO: 50 - DNA, 22 bp, Artificial sequence
  BRCA2 Primer Sequence
gtaatatcagttggcatttatt

SEQ ID NO: 51 - DNA, 21 bp, Artificial sequence
  BRCA2 Primer Sequence
tgcagaggtacatccaataag

SEQ ID NO: 52 - DNA, 21 bp, Artificial sequence
  BRCA2 Primer Sequence
gatcagtaaatagcaagtccg

SEQ ID NO: 53 - DNA, 23 bp, Artificial sequence
  BRCA2 Primer Sequence
tactgaaaatgaagataacaaat

SEQ ID NO: 54 - DNA, 22 bp, Artificial sequence
  BRCA2 Primer Sequence
attttgttctttcttatgtcag

SEQ ID NO: 55 - DNA, 35 bp, Artificial sequence
  BRCA2 Primer Sequence
tgtaaaacgacggccagtctactaaaacggagcaa

SEQ ID NO: 56 - DNA, 35 bp, Artificial sequence
  BRCA2 Primer Sequence
caggaaacagctatgaccgtatgaaaacccaacag

SEQ ID NO: 57 - DNA, 22 bp, Artificial sequence
  BRCA2 Primer Sequence
cacaaaatactgaaagaaagtg

SEQ ID NO: 58 - DNA, 19 bp, Artificial sequence
  BRCA2 Primer Sequence
ggcaccacagtctcaatag

SEQ ID NO: 59 - DNA, 20 bp, Artificial sequence
  BRCA2 Primer Sequence
gcaaagaccctaaagtacag

SEQ ID NO: 60 - DNA, 22 bp, Artificial sequence
  BRCA2 Primer Sequence
catcaaatattccttctctaag

SEQ ID NO: 61 - DNA, 35 bp, Artificial sequence
  BRCA2 Primer Sequence
tgtaaaacgacggccagtgaaaattcagccttagc

SEQ ID NO: 62 - DNA, 35 bp, Artificial sequence
  BRCA2 Primer Sequence
caggaaacagctatgaccatcagaatggtaggaat

SEQ ID NO: 63 - DNA, 22 bp, Artificial sequence
  BRCA2 Primer Sequence
gtactatagctgaaaatgacaa

SEQ ID NO: 64 - DNA, 20 bp, Artificial sequence
  BRCA2 Primer Sequence
accactggctatcctaaatg

SEQ ID NO: 65 - DNA, 20 bp, Artificial sequence
  BRCA2 Primer Sequence
tgaagatatttgcgttgagg

SEQ ID NO: 66 - DNA, 20 bp, Artificial sequence
  BRCA2 Primer Sequence
gtcagcaaaaaccttatgtg

SEQ ID NO: 67 - DNA, 21 bp, Artificial sequence
  BRCA2 Primer Sequence
acgaaaattatggcaggttgt

SEQ ID NO: 68 - DNA, 21 bp, Artificial sequence
  BRCA2 Primer Sequence
cttgtcttgcgttttgtaatg

SEQ ID NO: 69 - DNA, 20 bp, Artificial sequence
  BRCA2 Primer Sequence
gcttcataagtcagtctcat

SEQ ID NO: 70 - DNA, 20 bp, Artificial sequence
  BRCA2 Primer Sequence
tcaaattcctctaacactcc

SEQ ID NO: 71 - DNA, 35 bp, Artificial sequence
  BRCA2 Primer Sequence
tgtaaaacgacggccagttacagcaagtggaaagc

SEQ ID NO: 72 - DNA, 37 bp, Artificial sequence
  BRCA2 Primer Sequence
caggaaacagctatgaccaagtttcagttttaccaat

SEQ ID NO: 73 - DNA, 22 bp, Artificial sequence
  BRCA2 Primer Sequence
gttcttcagaaaataatcactc

SEQ ID NO: 74 - DNA, 21 bp, Artificial sequence
  BRCA2 Primer Sequence
tgtaaaaagagaatgtgtggc

SEQ ID NO: 75 - DNA, 39 bp, Artificial sequence
  BRCA2 Primer Sequence
tgtaaaacgacggccagtactttttctgatgttcctgtg

SEQ ID NO: 76 - DNA, 39 bp, Artificial sequence
  BRCA2 Primer Sequence
caggaaacagctatgacctaaaaatagtgattggcaaca

SEQ ID NO: 77 - DNA, 42 bp, Artificial sequence
  BRCA2 Primer Sequence
tgtaaaacgacggccagtagtggtgttttaaagtggtcaaaa

SEQ ID NO: 78 - DNA, 40 bp, Artificial sequence
  BRCA2 Primer Sequence
caggaaacagctatgaccggatccacctgaggtcagaata

SEQ ID NO: 79 - DNA, 21 bp, Artificial sequence
  BRCA2 Primer Sequence
taacatttaagcatccgttac

SEQ ID NO: 80 - DNA, 28 bp, Artificial sequence
  BRCA2 Primer Sequence
aaacgagacttttctcatactgtattag

SEQ ID NO: 81 - DNA, 22 bp, Artificial sequence
  BRCA2 Primer Sequence
accatgtagcaaatgagggtct

SEQ ID NO: 82 - DNA, 22 bp, Artificial sequence
  BRCA2 Primer Sequence
gcttttgtctgttttcctccaa

SEQ ID NO: 83 - DNA, 21 bp, Artificial sequence
  BRCA2 Primer Sequence
ccaggggttgtgctttttaaa

SEQ ID NO: 84 - DNA, 38 bp, Artificial sequence
  BRCA2 Primer Sequence
caggaaacagctatgaccactctgtcataaaagccatc

SEQ ID NO: 85 - DNA, 24 bp, Artificial sequence
  BRCA2 Primer Sequence
tttggtttgttataattgttttta

SEQ ID NO: 86 - DNA, 20 bp, Artificial sequence
  BRCA2 Primer Sequence
ccaactttttagttcgagag

SEQ ID NO: 87 - DNA, 19 bp, Artificial sequence
  BRCA2 Primer Sequence
ttcagtatcatcctatgtg

SEQ ID NO: 88 - DNA, 20 bp, Artificial sequence
  BRCA2 Primer Sequence
agaaaccttaacccatactg

SEQ ID NO: 89 - DNA, 39 bp, Artificial sequence
  BRCA2 Primer Sequence
tgtaaaacgacggccagtgaattctagagtcacacttcc

SEQ ID NO: 90 - DNA, 38 bp, Artificial sequence
  BRCA2 Primer Sequence
caggaaacagctatgacctttaactgaatcaatgactg

SEQ ID NO: 91 - DNA, 41 bp, Artificial sequence
  BRCA2 Primer Sequence
tgtaaaacgacggccagtaagtgaatatttttaaggcagtt

SEQ ID NO: 92 - DNA, 39 bp, Artificial sequence
  BRCA2 Primer Sequence
caggaaacagctatgaccaagagaccgaaactccatctc

SEQ ID NO: 93 - DNA, 38 bp, Artificial sequence
  BRCA2 Primer Sequence
tgtaaaacgacggccagtcactgtgcctggcctgatac

SEQ ID NO: 94 - DNA, 39 bp, Artificial sequence
  BRCA2 Primer Sequence
caggaaacagctatgaccatgttaaattcaaagtctcta

SEQ ID NO: 95 - DNA, 39 bp, Artificial sequence
  BRCA2 Primer Sequence
tgtaaaacgacggccagtgggtgttttatgcttggttct

SEQ ID NO: 96 - DNA, 40 bp, Artificial sequence
  BRCA2 Primer Sequence
caggaaacagctatgacccatttcaacatattccttcctg

SEQ ID NO: 97 - DNA, 19 bp, Artificial sequence
  BRCA2 Primer Sequence
aaccacacccttaagatga

SEQ ID NO: 98 - DNA, 20 bp, Artificial sequence
  BRCA2 Primer Sequence
gcattagtagtggattttgc

SEQ ID NO: 99 - DNA, 16 bp, Artificial sequence
  BRCA2 Primer Sequence
tcacttccattgcatc

SEQ ID NO: 100 - DNA, 17 bp, Artificial sequence
  BRCA2 Primer Sequence
tgccaactggtagctcc

SEQ ID NO: 101 - DNA, 20 bp, Artificial sequence
  BRCA2 Primer Sequence
tacagttagcagcgacaaaa

SEQ ID NO: 102 - DNA, 38 bp, Artificial sequence
  BRCA2 Primer Sequence
caggaaacagctatgaccatttgccaactggtagctcc

SEQ ID NO: 103 - DNA, 20 bp, Artificial sequence
  BRCA2 Primer Sequence
gctttcgccaaattcagcta

SEQ ID NO: 104 - DNA, 20 bp, Artificial sequence
  BRCA2 Primer Sequence
taccaaaatgtgtggtgatg

SEQ ID NO: 105 - DNA, 20 bp, Artificial sequence
  BRCA2 Primer Sequence
aatcactgatactggttttg

SEQ ID NO: 106 - DNA, 20 bp, Artificial sequence
  BRCA2 Primer Sequence
tatacttacaggagccacat

SEQ ID NO: 107 - DNA, 18 bp, Artificial sequence
  BRCA2 Primer Sequence
ctgtgtgtaatatttgcg

SEQ ID NO: 108 - DNA, 40 bp, Artificial sequence
  BRCA2 Primer Sequence
caggaaacagctatgacggcaagttcttcgtcagctattg

SEQ ID NO: 109 - DNA, 40 bp, Artificial sequence
  BRCA2 Primer Sequence
tgtaaaacgacggccagtgaattctcctcagatgactcca

SEQ ID NO: 110 - DNA, 38 bp, Artificial sequence
  BRCA2 Primer Sequence
caggaaacagctatgacctctttgctcattgtgcaaca

SEQ ID NO: 111 - DNA, 1158 bp, Homo sapiens
  misc_feature (1)..(1158): Exon 10; Nucleotides 31-1146 correspond to nucleotides 1022-2137 of complete coding sequence
ttaatgtgcttctgttttatactttaacaggatttggaaaaacatcagggaattcatttaaagtaaatagctgcaaagaccacattggaaagtcaatgccamatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcamatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcrgagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacattttataaaggaaaaaaaataccgaaagaccaaaaatcagaactaattaactgttcagcccagtttgaagcaaatgcttttgaagcaccacttacatttgcaaatgctgattcaggtacctctgtct

SEQ ID NO: 112 - DNA, 4987 bp, Homo sapiens
  misc_feature (1)..(4987): Exon 11, nucleotides 20-4951 correspond to nucleotides 2138-7069 of complete coding sequence
tttgtgtttttatgtttaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacaytcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaartagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgracaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaarccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgtygtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctgttgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtctcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggtaagtgttcatttttacctttcgtgttgccaatca

SEQ ID NO: 113 - DNA, 468 bp, Homo sapiens
  misc_feature (1)..(468): Exon 14, nucleotides 12-439 correspond to nucleotides 7236-7663 of complete coding sequence
ccccattgcagcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacrttttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagcagctgtaactttcacaaagtgtgaagaagaacctttaggtattgtatgacaatttgtgtgatgaatt

SEQ ID NO: 114 - DNA, 255 bp, Homo sapiens
  misc_feature (1)..(255): Exon 22, nucleotides 31-229 correspond to nucleotides 8983-9181 of complete coding sequence
tttttattccaatatcttaaatggtcacagggttatttcagtgaagagcagttaagagccttgaataatcacaggcaaatgttgaatgataagaaacaagctcagatccagttggaaattaggaagrccatggaatctgctgaacaaaaggaacaaggtttatcaagggatgtcacaaccgtgtggaagttgcgtattgtaagctattcaaaaaaagaaaaagattcaggtaagtatgtaaatgctttgttttta

SEQ ID NO: 115 - DNA, 157 bp, Homo sapiens
  misc_feature (31)..(135): Exon 2
taagtgcattttggtcttctgttttgcagacttatttaccaagcattggaggaatatcgtaggtaaaaatgcctattggatccaaagagaggccaacattttttgaaatttttaagacacgctgcaacaaagcaggtattgacaaattttatataac

SEQ ID NO: 116 - DNA, 297 bp, Homo sapiens
  misc_feature (21)..(269): Exon 3
gggattttttttttaaatagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggtaagtaatgcaatatggtagactgggg

SEQ ID NO: 117 - DNA, 159 bp, Homo sapiens
  misc_feature (26)..(134): Exon 4
tcactgaattattgtactgtttcaggaaggaatgttcccaatagtagacataaaagtcttcgcacagtgaaaactaaaatggatcaagcagatgatgtttcctgtccacttctaaattcttgtcttagtgaaaggtatgatgaagctattatattaaaa

SEQ ID NO: 118 - DNA, 80 bp, Homo sapiens
  misc_feature (31)..(71): Exon 6
ttaacaattttcccctttttttacccccagtggtatgtgggagtttgtttcatacaccaaagtttgtgaaggtaaatatt

SEQ ID NO: 119 - DNA, 174 bp, Homo sapiens
  misc_feature (51)..(165): Exon 7
taatgatcagggcatttctataaaaaataaactattttctttcctcccagggtcgtcagacaccaaaacatatttctgaaagtctaggagctgaggtggatcctgatatgtcttggtcaagttctttagctacaccacccacccttagttctactgtgctcataggtaataata

SEQ ID NO: 120 - DNA, 90 bp, Homo sapiens
  misc_feature (14)..(63): Exon 8
ttttatcttacagtcagaaatgaagaagcatctgaaactgtatttcctcatgatactactgctgtaagtaaatatgacattgattagact

SEQ ID NO: 121 - DNA, 142 bp, Homo sapiens
  misc_feature (20)..(131): Exon 9
taaactataatttttgcagaatgtgaaaagctatttttccaatcatgatgaaagtctgaagaaaaatgatagatttatcgcttctgtgacagacagtgaaaacacaaatcaaagagaagctgcaagtcatggtaagtcctct

SEQ ID NO: 122 - DNA, 147 bp, Homo sapiens
  misc_feature (29)..(124): Exon 12
aaaacatatatgaaatatttctttttaggagaaccctcaatcaaaagaaacttattaaatgaatttgacaggataatagaaaatcaagaaaaatccttaaaggcttcaaaaagcactccagatggtaaaattagctttttatttata

SEQ ID NO: 123 - DNA, 125 bp, Homo sapiens
  misc_feature (31)..(100): Exon 13
aatatgtaatataaaataattgtttcctaggcacaataaaagatcgaagattgtttatgcatcatgtttctttagagccgattacctgtgtaccctttcggtaagacatgtttaaatttttctaa

SEQ ID NO: 124 - DNA, 199 bp, Homo sapiens
  misc_feature (13)..(183): Exon 17
ttatttgttcagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacaggcaagtttaaagcatt

SEQ ID NO: 125 - DNA, 380 bp, Homo sapiens
  misc_feature (19)..(373): Exon 18
ttttgttttcacttttagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaaggtaaatt

SEQ ID NO: 126 - DNA, 208 bp, Homo sapiens
  misc_feature (30)..(185): Exon 19
taaatcaatatatttattaatttgtccagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacaggtatgatgtattcttgaaactta

SEQ ID NO: 127 - DNA, 206 bp, Homo sapiens
  misc_feature (28)..(172): Exon 20
tttggtgtgtgtaacacattattacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaaggtaaaattagttatatggtacacattgttatttc

SEQ ID NO: 128 - DNA, 185 bp, Homo sapiens
  misc_feature (36)..(157): Exon 21
agtttagtgaattaataatccttttgttttcttagaaaacacaacaaaaccatatttaccatcacgtgcactaacaagacagcaagttcgtgctttgcaagatggtgcagagctttatgaagcagtgaagaatgcagcagacccagcttaccttgaggtgagagagtaagaggacatataatgag

SEQ ID NO: 129 - DNA, 200 bp, Homo sapiens
  misc_feature (12)..(175): Exon 23
tctccaaacagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtacaaacctttcattgtaattttt

SEQ ID NO: 130 - DNA, 217 bp, Homo sapiens
  misc_feature (25)..(163): Exon 24
gaatttttgttttgttttctgtaggtttcagatgaaattttatttcagatttaccagccacgggagccccttcacttcagcaaatttttagatccagactttcagccatcttgttctgaggtggacctaataggatttgtcgtttctgttgtgaaaaaaacaggtaatgcacaatatagttaattttttttattgattcttttaaaaaacattgtct

SEQ ID NO: 131 - DNA, 284 bp, Homo sapiens
  misc_feature (31)..(275): Exon 25
taacattcttttcttttttttccattctaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgaggtaaggtta

SEQ ID NO: 132 - DNA, 210 bp, Homo sapiens
  misc_feature (31)..(177): Exon 26
ataaagcagcttttccacttattttcttagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctggtaagttaatgtaaactcaaggaatattataag

SEQ ID NO: 133 - DNA, 691 bp, Homo sapiens
  misc_feature (23)..(691): Exon 27
tacgttttcatttttttatcagatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaagcatttgcaaaggcgacaataaattattgacgcttaacctttccagtttataagactgga

SEQ ID NO: 134 - DNA, 10987 bp, Homo sapiens
  misc_feature: GenBank Accession No. U43746, BRCA2 gene coding sequence 229...10485
ggtggcgcgagcttctgaaactaggcggcagaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcacatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacattttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctgttgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtctcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagcagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaagcatttgcaaaggcgacaataaattattgacgcttaacctttccagtttataagactggaatataatttcaaaccacacattagtacttatgttgcacaatgagaaaagaaattagtttcaaatttacctcagcgtttgtgtatcgggcaaaaatcgttttgcccgattccgtattggtatacttttgcttcagttgcatatcttaaaactaaatgtaatttattaactaatcaagaaaaacatctttggctgagctcggtggctcatgcctgtaatcccaacactttgagaagctgaggtgggaggagtgcttgaggccaggagttcaagaccagcctgggcaacatagggagacccccatctttacgaagaaaaaaaaaaaggggaaaagaaaatcttttaaatctttggatttgatcactacaagtattattttacaatcaacaaaatggtcatccaaactcaaacttgagaaaatatcttgctttcaaattgacacta

SEQ ID NO: 135 - DNA, 10 bp, Artificial sequence
  correct 3′ end of BRCA2 intron 4
tttattttag

SEQ ID NO: 136 - DNA, 16 bp, Artificial sequence
  correct 3′ end of BRCA2 exon 5
gtgttctcataaacag

(19) United States
(12) Patent Application Publication
(10) Pub. No.: US 2009/0325237 A1
(43) Pub. Date: Dec. 31, 2009

(54) Method of Analyzing a BRCA2 Gene in a Human Subject

(75) Inventors: Patricia D. Murphy, Slingerlands, NY (US); Marga B. White, Frederick, MD (US); Mark B. Rabin, Rockville, MD (US); Sheri J. Olson, Falls Church, VA (US); Matthew Yoshikawa, Germantown, MD (US); Geoffrey M. Jackson, Beltsville, MD (US); Tara Eskandari, Rockville, MD (US); Brenda Schryer, Bel Air, MD (US); Michael Park, Rockville, MD (US)

Correspondence Address: MORGAN LEWIS & BOCKIUS LLP, 1111 PENNSYLVANIA AVENUE NW, WASHINGTON, DC, 20004, US

(73) Assignee: Ore Pharmaceuticals Inc., Gaithersburg, MD (US)

(21) Appl. No.: 12/466,490

(22) Filed: May 15, 2009

Related U.S. Application Data
(63) Continuation of application No. 12/402,298, filed on Mar. 11, 2009.
Application No. 12/402,298 is a continuation of application No. 11/267,381, filed on Nov. 7, 2005.
Application No. 11/267,381 is a continuation of application No. 09/084,471, filed on May 22, 1998.
(60) Provisional application No. 60/055,784, filed on Aug. 15, 1997.
(60) Provisional application No. 60/064,926, filed on Nov. 7, 1997.
(60) Provisional application No. 60/065,367, filed on Nov. 12, 1997.

(51) Int. Cl. C12P 19/34; C07H 21/04

(52) U.S. Cl. 435/91.2; 536/23.1

(57) ABSTRACT

Five novel DNA and protein sequences have been determined for the BRCA2 gene, as have been ten polymorphic sites and their rates of occurrence in the normal alleles of BRCA2. The sequences BRCA2^{(omi 1-5)} and the ten polymorphic sites will provide greater accuracy and reliability for genetic testing. One skilled in the art will be better able to avoid misinterpretations of changes in the gene and/or protein sequence, determine the presence of a normal sequence, and of mutations of BRCA2. This invention is also related to a method of performing gene therapy with BRCA2^{(omi 1-5)} coding sequences or fragments thereof. This invention is further related to protein therapy with BRCA2^{(omi 1-5)} proteins or their functional equivalents.

This is an U.S. utility patent application based on U.S. Provisional Application Ser. Nos. 60/055,784 filed on Aug. 15, 1997, 60/064,926 filed on Nov. 7, 1997, and 60/065,367 filed on Nov. 12, 1997.

### FIELD OF THE INVENTION

This invention relates to a gene which has been associated with breast cancer where the gene is found to be mutated. More specifically, this invention relates to five unique coding sequences of BRCA2 gene BRCA2^{(omi1)}, BRCA2^{(omi2)}, BRCA2^{(omi3)}, BRCA2^{(omi4)}, and BRCA2^{(omi5)} identified in human subjects which define five novel haplotypes.

### BACKGROUND OF THE INVENTION

It has been estimated that about 5-10% of breast cancer is inherited (Rowell, S., et al., American Journal of Human Genetics 55:861-865 (1994)). The first gene associated with both breast and ovarian cancer was cloned in 1994 from chromosome 17 by Miki, Y., et al., Science 266:66-71 (1994). A second high-risk breast cancer conferring gene was located on chromosome 13 in 1994 (Wooster, R., et al., Science 265:2088-2090) and subsequently cloned in 1995 (Wooster, R., et al., Nature 378:789-792). Mutations in this “tumor suppressor” gene are thought to account for roughly 35% of inherited breast cancer and 80-90% of families with male breast cancer.

Locating one or more mutations in the BRCA2 region of chromosome 13 provides a promising approach to reducing the high incidence and mortality associated with breast cancer through the early detection of women and men at high risk. These individuals, once identified, can be targeted for more aggressive prevention programs. Screening is carried out by a variety of methods which include karyotyping, probe binding and DNA sequencing.

In DNA sequencing technology, genomic DNA is extracted from whole blood and the coding regions of the BRCA2 gene are amplified. Each of the coding regions may be sequenced completely and the results are compared to the normal DNA sequence of the gene. Alternatively, the coding sequence of the sample gene may be compared to a panel of known mutations or other screening procedure before completely sequencing the gene and comparing it to a normal sequence of the gene.

The BRCA2 gene is divided into 27 separate exons. Exon 1 is noncoding, in that it is not part of the final functional BRCA2 protein product. The BRCA2 coding region spans roughly 10433 base pairs (bp) over 70 kb. Each exon consists of 100-600 bp, except for exons 10, 11 and 27. The full length mRNA is 11-12 kb. To sequence the coding region of the BRCA2 gene, each exon is amplified separately and the resulting PCR products are sequenced in the forward and reverse directions. Because exons 10, 11, and 27 are so large, we have divided them into three, twenty-one, and two overlapping PCR fragments (respectively) of approximately 250-625 bp each (segments “A” through “C” of exon 10, “A” through “U” of exon 11, and “A” through “B” of exon 27).

Many mutations and normal polymorphisms have already been reported in the BRCA2 gene. A world wide web site has been built to facilitate the detection and characterization of alterations in breast cancer susceptibility genes. Such mutations in BRCA2 can be accessed through the Breast Cancer Information Core (BIC) at http://www.nhgri.nih.gov/Intramural_research/Lab_transfer/Bic. This data site became publicly available on Nov. 1, 1995. Friend, S. et al. Nature Genetics 11:238, (1995). The information on BRCA2 was added in February, 1996.

The genetics of Breast Cancer Syndrome is autosomal dominant with reduced penetrance. In simple terms, this means that the syndrome runs through families: (1) both sexes can be carriers (mostly women get the disease but men can both pass it on and occasionally get breast cancer); (2) most generations will likely have breast cancer; (3) occasionally women carriers either die young before they have the time to manifest disease (and yet have offspring who get it) or they never develop breast or ovarian cancer and die of old age (the latter people are said to have “reduced penetrance” because they never develop cancer). Pedigree analysis and genetic counseling is absolutely essential to the proper workup of a family prior to any lab work.

Until now, the only sources of genomic sequence information for BRCA2 were GenBank (Accession Number U43746), or through the Breast Information Core (BIC) database on the Internet which requires membership in the BIC consortium. However, based upon the disclosure of this patent application, in neither GenBank nor BIC were the sequences identified and listed entirely accurate. There is a need in the art to correct these mistakes which otherwise may lead to misinterpretation of the sequence data from the patient as abnormal when it was not, or vice versa.

In addition, there is a need in the art to have available a functional allele profile which represents the most likely BRCA2 sequences to be found in the majority of the normal population. This functional allele profile is based upon frequent polymorphisms and the correct backbone sequence. The knowledge of several common normal haplotypes will make it possible for true mutations to be easily identified or differentiated from polymorphisms. Identification of mutations of the BRCA2 gene and protein would allow more widespread diagnostic screening for hereditary breast cancer than is currently possible.

The use of these common normal haplotypes, in addition to the previously published BRCA2 sequence, will reduce the likelihood of misinterpreting a “sequence variation” found in the normal population with a pathologic “mutation” (i.e. causes disease in the individual or puts the individual at a high risk of developing the disease). With large interest in breast cancer predisposition testing, misinterpretation is particularly worrisome. People who already have breast cancer are asking the clinical question: “is my disease caused by a heritable genetic mutation?” The relatives of the those with breast cancer are asking the question: “Am I also a carrier of the mutation my relative has? Thus, is my risk increased, and should I undergo a more aggressive surveillance program?”

### SUMMARY OF THE INVENTION

The present invention is based on the discovery of the correct genomic BRCA2 sequence and five novel sequence haplotypes found in normal human subjects of the BRCA2 gene.

It is an object of this invention to provide the correct intronic/exonic sequence of the BRCA2 gene.

It is another object of this invention to provide five unique haplotype sequences of the BRCA2 gene in normal individuals which do not correspond to increased cancer susceptibility.

It is another object of this invention to sequence a BRCA2 gene or a portion thereof and compare it to the five haplotype sequences to determine whether a sequence variation noted represents a polymorphism or a potentially harmful mutation.

It is another object of this invention to provide a list of the pairs which occur at each of ten polymorphic points in the BRCA2 gene.

It is another object of this invention to provide the rates of occurrence for the polymorphisms at codons 289, 372, 455, 743, 894, 991, 1132, 1269, 2414, and 2951 in the BRCA2 gene.

It is another object of this invention to provide a method wherein all exons of BRCA2 gene or parts thereof, are amplified with one or more oligonucleotide primers.

It is another object of this invention to provide a method of identifying a individual who carries no mutation(s) of the BRCA2 gene and is therefore at no increased risk or susceptibility to breast or ovarian cancer based on a finding that the individual does not carry an abnormal BRCA2 genes.

It is another object of this invention to provide a method of identifying a mutation in BRCA2 gene leading to predisposition or higher susceptibility to breast or ovarian cancer.

It is another object of this invention to provide five novel BRCA2 protein sequences derived from five BRCA2 haplotype sequences.

It is another object of the invention to encompass prokaryotic or eukaryotic host cells comprising an expression vector having a DNA sequence that encodes for all or a fragment of the five novel BRCA2 protein sequences, a BRCA2 polypeptide thereof, or a functional equivalent thereof.

It is another object of the invention to encompass an anti-BRCA2 protein antibody using all of fragments of the five novel BRCA2 protein sequences, a BRCA2 polypeptide thereof or a functional equivalent thereof as an immunogen.

There is a need in the art for cDNA sequences of the BRCA2 gene and for the protein sequences of BRCA2 gene from normal individuals who are not at risk for increased susceptibility for cancer. In order to determine whether a sample from a patient suspected of containing a BRCA2 mutation actually has the mutation, the patient's BRCA2 DNA and/or amino acid sequence need to be compared to all known normal BRCA2 sequences. Failure to compare the sequence obtained to all naturally occurring normal sequences may result in reporting a sample as containing a potentially harmful mutation when it is a polymorphism without clinical significance.

A person skilled in the art of genetic susceptibility testing will find the present invention useful for:
a) identifying individuals having a normal BRCA2 gene with no coding sequence mutations, who therefore cannot be said to have an increased genetic susceptibility to breast or ovarian cancer from their BRCA2 genes;
b) avoiding misinterpretation of normal polymorphisms found in the BRCA2 gene;
c) determining the presence of a previously unknown mutation in the BRCA2 gene;
d) identifying a mutation in exon 11 of BRCA2 which indicates a predisposition or higher susceptibility to ovarian cancer than breast cancer (i.e., resides in the putative “ovarian cancer cluster” region);
e) probing a human sample of the BRCA2 gene by allele to determine the presence or absence of either polymorphic alleles or mutations;
f) performing gene therapy with the correct BRCA2 gene sequence.
g) performing protein replacement therapy with the correct BRCA 2 protein sequence or a functional equivalent thereof.

### BRIEF DESCRIPTION OF THE FIGURES

FIG. 1 shows the GenBank genomic sequence of BRCA2 (Accession Number U43746). The lower case letters denote intronic sequences and the upper case letters denote exonic sequences. Incorrect exonic sequences at exons 5 and 16 are shown with boldface type.

FIG. 2 shows the corrected genomic sequence of BRCA2. The lower case letters denote intronic sequences and the upper case letters denote exonic sequences. Corrected intronic and exonic sequences at exons 5, 11 and 15 are shown with boldface type.

FIG. 3 shows the alternative alleles at polymorphic sites along a chromosome which can be represented as a unit or “haplotype” within a gene such as BRCA2. The haplotype that is in GenBank (GB) is shown with light shading. Five additional haplotypes are shown in FIG. 3 (encompassing the alternative alleles found at nucleotide sites 1093, 1342, 1593, 2457, 2908, 3199, 3624, 4035, 7470 and 9079). BRCA2^{(omi-1)}, BRCA2^{(omi-2)}, BRCA2^{(omi-4)}, BRCA2^{(omi-4)}, and BRCA2^{(omi-5)} are represented with mixed dark and light shading (numbers 2, 4, 6, 8 and 10 from left to right). In total, 5 of 10 haplotypes along the BRCA2 gene are unique.

### DETAILED DESCRIPTION OF THE INVENTION

### Definitions

The following definitions are provided for the purpose of understanding this invention.

“Breast and Ovarian cancer” is understood by those skilled in the art to include breast, ovarian and pancreatic cancer in women and also breast, prostate and pancreatic cancer in men. BRCA2 is associated with genetic susceptibility to breast, ovarian and pancreatic cancer. Therefore, claims in this document which recite breast and/or ovarian cancer refer to breast, ovarian, prostate, and pancreatic cancers in men and women.

“Coding sequence” refers to those portions of a gene which, taken together, code for a peptide (protein), or which nucleic acid itself has function.

“Protein” or “peptide” refers to a sequence of amino acids which has function.

“BRCA2^{(omi)}” refers to the genomic BRCA2 sequence disclosed in Genbank (Accession Number U43746) wherein,
(1) a 10 bp stretch (5′-TTTATTTTAG-3′) is intronic at 3′ end of intron 4, rather than at the 5′ end of exon 5; and
(2) a 16 bp stretch (5′-GTGTTCTCATAAACAG-3′) is exonic at the 3′ end of exon 15, rather than at the 5′ end of exon.

“BRCA2^{(omi 1-5)}” refers to five unique DNA sequences of the BRCA2 gene and their introns (particularly the slice sites adjacent to the exons). These sequences were found by end to end sequencing of the BRCA2 gene from 5 individuals randomly drawn from the population and who were documented to have no family history of breast or ovarian cancer. The sequenced exons were found not to contain any truncating mutations. In all cases the change of a nucleic acid at a polymorphic site lead to a codon change and a change of amino acid from the previously published standard in GenBank (see TABLE III). In some cases the frequency of occurrence of a nucleic acid change was found to differ from the published frequency or was newly determined. These sequence variations are believed to be alleles whose haplotypes do not indicate an increased risk for cancer.

“Normal DNA sequence” also called “normal gene sequence” refers to a nucleic acid sequence, the nucleic acid of which are known to occur at their respective positions with high frequency in a population of individuals who carry the gene which codes for a normally functioning protein, or which itself has normal function.

“Normal Protein Sequence” refers to the protein sequence, the amino acids of which are known to occur with high frequency in a population of individuals who carry the gene which codes for a normally functioning protein.

“Normal Sequence” refers to the nucleic acid or protein sequence, the nucleic or amino acids of which are known to occur with high frequency in a population of individuals who carry the gene which codes for a normally functioning protein, or which nucleic acid itself has a normal function.

“Haplotype” refers to a series of specific alleles within a gene along a chromosome.

“Functional allele profile” refers a list of those alleles in the normal population which have the funII function.

“Mutation” refers to a base change or a gain or loss of base pair(s) in a DNA sequence, which results in a DNA sequence coding for a non-functional protein or a protein with substantially reduced or altered function.

“Polymorphism” refers to a base change in a DNA sequence which is not associated with known pathology.

“Primer” refers to a sequence comprising about 15 or more nucleotides having a sequence complementary to the BRCA2 gene. Other primers which can be used for primer hybridization will be known or readily ascertainable to those skilled in the art.

“Substantially complementary to” refers to primer sequences which hybridize to the sequences provided under stringent conditions and/or sequences having sufficient homology with BRCA2 sequences, such that the allele specific oligonucleotide primers hybridize to the BRCA2 sequences to which they are complimentary.

“Isolated nucleic acids” refers to nucleic acids substantially free of other nucleic acids, proteins, lipids, carbohydrates or other materials with which they may be associated. Such association is typically either in cellular material or in a synthesis medium.

“Biological sample” or “body sample” refers to a sample containing DNA obtained from a biological source. The sample may be from a living, dead or even archeological source from a variety of tissues and cells. Examples include body fluid (e.g. blood (leukocytes), urine (epithelial cells), saliva, breast milk, menstrual flow, cervical and vaginal secretions, etc.), skin, hair roots/follicle, mucus membrane (e.g. buccal or tongue cell scrapings), cervicovaginal cells (from PAP smear, etc.), lymphatic tissue, internal tissue (normal or tumor).

“Vector” refers to any polynucleotide which is capable of self replication or inducing integration into a self-replicating polynucleotide. Examples include polynucleotides containing an origin or replication or an integration site. Vectors may be intergrated into the host cell's chromosome or form an autonomously replicating unit.

“A tumor growth inhibitor” refers to a molecule such as, all or a fragment of BRCA2 protein, a BRCA2 polypeptide, or a functional equivalent thereof that is effective for preventing the formation of, reducing, or eliminating a transformed or malignant phenotype of breast or ovarian cancer cells.

“A BRCA2 polypeptide” refers to a BRCA2 polypeptide either directly derived from the BRCA2 protein, or homologous to the BRCA2 protein, or a fusion protein consisting of all or fragments of the BRCA2 protein and polypeptides.

“A functional equivalent” refers to a molecule including an unnatural BRCA2 polypeptide, a drug or a natural product which retains substantial biological activity as the native BRCA2 protein. The activity and function of BRCA2 protein may include transactivation, granin, DNA repair, among others.

“A target polynucleotide” refers to the nucleic acid sequence of interest, for example, the BRCA2 encoding polynucleotide. Other primers which can be used for primer hybridization will be known or readily ascertainable to those of skill in the art.

The invention in several of its embodiments includes: an isolated DNA sequence of the BRCA2 coding sequence as set forth in SEQ ID NO:4, 6, 8, 10, and 12, a protein sequence of the BRCA2 protein as set forth in SEQ ID NO:5, 7, 9, 11, 13, a method of identifying individuals having a normal BRCA2 gene with no increased risk for breast and ovarian cancer, a method of detecting an increased genetic susceptibility to breast and ovarian cancer in an individual resulting from the presence of a mutation in the BRCA2 coding sequence, a method of performing gene therapy to prevent or treat a tumor, a method of protein replacement therapy to prevent or treat a tumor, a diagnostic reagent comprising all or fragments of the disclosed BRCA2 cDNA and protein sequences.

### Sequencing

Any nucleic acid specimen, in purified or non-purified form, can be utilized as the starting nucleic acid, providing it contains, or is suspected of containing, the specific nucleic acid sequence containing a polymorphic or a mutant allele. Thus, the process may amplify, for example, DNA or RNA, including mRNA and cDNA, wherein DNA or RNA may be single stranded or double stranded. In the event that RNA is to be used as a template, enzymes and/or conditions optimal for reverse transcribing the template to DNA would be utilized. In addition, a DNA-RNA hybrid which contains one strand of each may be utilized. A mixture of nucleic acids may also be employed, or the nucleic acids produced in a previous method such as an amplification reaction using the same or different primers may be so utilized. The specific nucleic acid sequence to be amplified, i.e., the polymorphic and/or the mutant allele, may be a fraction of a larger molecule or can be present initially as a discrete molecule, so that the specific sequence constitutes the entire nucleic acid. A variety of amplification techniques may be used such as ligating the DNA sample or fragments thereof to a vector capable of replication or incorporation into a replicating system thereby increasing the number of copies of DNA suspected of containing at least a portion of the BRCA2 gene. Amplification techniques include so called “shot gun cloning”. It is not necessary that the sequence to be amplified be present initially in a pure form; it may be a minor fraction of a complex mixture, such as contained in whole human DNA.

It should be noted that one need not sequence the entire coding region or even an entire DNA fragment in order to determine whether or not a mutation is present. For example, when a mutation is known in one family member, it is sufficient to determine the sequence at only the mutation site by sequencing or by other mutation detection systems such as ASO when testing other family members.

DNA utilized herein may be extracted from a body sample, such as blood, tissue material and other biological sample by a variety of techniques such as that described by Maniatis, et al. in Molecular Cloning: A Laboratory Manual, Cold Spring Harbor, N.Y., p 280-281, 1982). If the extracted sample is impure, it may be treated before amplification with an amount of a reagent effective to open the cells, and to expose and/or separate the strand(s) of the nucleic acid(s). This lysing and nucleic acid denaturing step to expose and separate the strands will allow amplification to occur much more readily.

For amplification by cloning, the isolated DNA may be cleaved into fragments by a restriction endonuclease or by shearing by passing the DNA containing mixture through a 25 gauge needle from a syringe to prepare 1-1.5 kb fragments. The fragments are then ligated to a cleaved vector (virus, plasmid, transposon, cosmid etc.) and then the recombinant vector so formed is then replicated in a manner typical for that vector.

For a PCR amplification, the deoxyribonucleotide triphosphates dATP, dCTP, dGTP, and dTTP are added to the synthesis mixture, either separately or together with the primers, in adequate amounts and the resulting solution is heated to about 90°-100° C. from about 1 to 10 minutes, preferably from 1 to 4 minutes. After this heating period, the solution is allowed to cool, which is preferable for the primer hybridization. To the cooled mixture is added an appropriate agent for effecting the primer extension reaction (called herein “agent for polymerization”), and the reaction is allowed to occur under conditions known in the art. The agent for polymerization may also be added together with the other reagents if it is heat stable. This synthesis (or amplification) reaction may occur at room temperature up to a temperature above which the agent for polymerization no longer functions. Thus, for example, if DNA polymerase is used as the agent, the temperature is generally no greater than about 40° C. Most conveniently the reaction occurs at room temperature. When using thermostable DNA polymerase such as Taq, higher temperature may be used.

The allele specific oligonucleotide primers are useful in determining whether a subject is at risk of having breast or ovarian cancer, and also useful for characterizing a tumor. Primers direct amplification of a target polynucleotide prior to sequencing. These unique BRCA2 oligonucleotide primers for exons 2-27 shown in TABLE II were designed and produced specifically to optimize amplification of portions of BRCA2 which are to be sequenced.

The primers used to carry out this invention embrace oligonucleotides of sufficient length and appropriate sequence to provide initiation of polymerization. Environmental conditions conducive to synthesis include the presence of nucleoside triphosphates and an agent for polymerization, such as DNA polymerase, and a suitable temperature and pH. The primer is preferably single stranded for maximum efficiency in amplification, but may be double stranded. If double stranded, the primer is first treated to separate its strands before being used to prepare extension products. The primer must be sufficiently long to prime the synthesis of extension products in the presence of the inducing agent for polymerization. The exact length of primer will depend on many factors, including temperature, buffer, and nucleotide composition. The oligonucleotide primer typically contains 18-28 bp plus in some cases an M13 “tail” for convenience.

Primers used to carry out this invention are designed to be substantially complementary to each strand of the genomic locus to be amplified. This means that the primers must be sufficiently complementary to hybridize with their respective strands under conditions which allow the agent for polymerization to perform. In other words, the primers should have sufficient complementarity with the 5′ and 3′ sequences flanking the mutation to hybridize therewith and permit amplification of the genomic locus.

Oligonucleotide primers of the invention are employed in the amplification process which is an enzymatic chain reaction that produces exponential quantities of polymorphic locus relative to the number of reaction steps involved. Typically, one primer is complementary to the negative (−) strand of the polymorphic locus and the other is complementary to the positive (+) strand. Annealing the primers to denatured nucleic acid followed by extension with an enzyme, such as the large fragment of DNA polymerase I (Klenow) and nucleotides, results in newly synthesized + and − strands containing the target polymorphic locus sequence. Because these newly synthesized sequences are also templates, repeated cycles of denaturing, primer annealing, and extension results in exponential production of the region (i.e., the target polymorphic locus sequence) defined by the primers. The product of the chain reaction is a discreet nucleic acid duplex with termini corresponding to the ends of the specific primers employed.

The oligonucleotide primers of the invention may be prepared using any suitable method, such as conventional phosphotriester and phosphodiester methods or automated embodiments thereof. In one such automated embodiment, diethylphosphoramidites are used as starting materials and may be synthesized as described by Beaucage, et al., Tetrahedron Letters, 22:1859-1862, 1981. One method for synthesizing oligonucleotides on a modified solid support is described in U.S. Pat. No. 4,458,066.

The agent for polymerization may be any compound or system which will function to accomplish the synthesis of primer extension products, including enzymes. Suitable enzymes for this purpose include, for example, E. coli DNA polymerase I, Klenow fragment of E. coli DNA polymerase, polymerase muteins, reverse transcriptase, other enzymes, including heat-stable enzymes (i.e., those enzymes which perform primer extension after being subjected to temperatures sufficiently elevated to cause denaturation), such as Taq polymerase. Suitable enzymes will facilitate combination of the nucleotides in the proper manner to form the primer extension products which are complementary to each polymorphic locus nucleic acid strand. Generally, the synthesis will be initiated at the 3′ end of each primer and proceed in the 5′ direction along the template strand, until synthesis terminates, producing molecules of different lengths.

The newly synthesized strand and its complementary nucleic acid strand will form a double-stranded molecule under hybridizing conditions described above and this hybrid is used in subsequent steps of the process. In the next step, the newly synthesized double-stranded molecule is subjected to denaturing conditions using any of the procedures described above to provide single-stranded molecules.

The steps of denaturing, annealing, and extension product synthesis can be repeated as often as needed to amplify the target polymorphic locus nucleic acid sequence to the extent necessary for detection. The amount of the specific nucleic acid sequence produced will accumulate in an exponential fashion. Amplification is described in PCR. A Practical Approach, ILR Press, Eds. M. J. McPherson, P. Quirke, and G. R. Taylor, 1992.

The amplification products may be detected by Southern blots analysis, without using radioactive probes. In such a process, for example, a small sample of DNA containing a very low level of the nucleic acid sequence of the polymorphic locus is amplified, and analyzed via a Southern blotting technique or similarly, using dot blot analysis. The use of non-radioactive probes or labels is facilitated by the high level of the amplified signal. Alternatively, probes used to detect the amplified products can be directly or indirectly detectably labeled, for example, with a radioisotope, a fluorescent compound, a bioluminescent compound, a chemiluminescent compound, a metal chelator or an enzyme. Those of ordinary skill in the art will know of other suitable labels for binding to the probe, or will be able to ascertain such, using routine experimentation.

Sequences amplified by the methods of the invention can be further evaluated, detected, cloned, sequenced, and the like, either in solution or after binding to a solid support, by any method usually applied to the detection of a specific DNA sequence such as PCR, oligomer restriction (Saiki, et. al., Bio/Technology, 3:1008-1012, 1985), allele-specific oligonucleotide (ASO) probe analysis (Conner, et al., Proc. Natl. Acad. Sci. U.S.A., 80:278, 1983), oligonucleotide ligation assays (OLAs) (Landgren, et al., Science, 241:1007, 1988), and the like. Molecular techniques for DNA analysis have been reviewed (Landgren, et al., Science, 242:229-237, 1988).

Preferably, the method of amplifying is by PCR, as described herein and as is commonly used by those of ordinary skill in the art. Alternative methods of amplification have been described and can also be employed as long as the BRCA2 locus amplified by PCR using primers of the invention is similarly amplified by the alternative means. Such alternative amplification systems include but are not limited to self-sustained sequence replication, which begins with a short sequence of RNA of interest and a T7 promoter. Reverse transcriptase copies the RNA into cDNA and degrades the RNA, followed by reverse transcriptase polymerizing a second strand of DNA. Another nucleic acid amplification technique is nucleic acid sequence-based amplification (NASBA) which uses reverse transcription and T7 RNA polymerase and incorporates two primers to target its cycling scheme. NASBA can begin with either DNA or RNA and finish with either, and amplifies to 10⁸ copies within 60 to 90 minutes. Alternatively, nucleic acid can be amplified by ligation activated transcription (LAT). LAT works from a single-stranded template with a single primer that is partially single-stranded and partially double-stranded. Amplification is initiated by ligating a cDNA to the promoter oligonucleotide and within a few hours, and amplification is 10⁸ to 10⁹ fold. Another amplification system useful in the method of the invention is the Qβ Replicase System. The Qβ replicase system can be utilized by attaching an RNA sequence called MDV-1 to RNA complementary to a DNA sequence of interest. Upon mixing with a sample, the hybrid RNA finds its complement among the specimen's mRNAs and binds, activating the replicase to copy the tag-along sequence of interest. Another nucleic acid amplification technique, ligase chain reaction (LCR), works by using two differently labeled halves of a sequence of interest which are covalently bonded by ligase in the presence of the contiguous sequence in a sample, forming a new target. The repair chain reaction (RCR) nucleic acid amplification technique uses two complementary and target-specific oligonucleotide probe pairs, thermostable polymerase and ligase, and DNA nucleotides to geometrically amplify targeted sequences. A 2-base gap separates the oligonucleotide probe pairs, and the RCR fills and joins the gap, mimicking normal DNA repair. Nucleic acid amplification by strand displacement activation (SDA) utilizes a short primer containing a recognition site for hincII with short overhang on the 5′ end which binds to target DNA. A DNA polymerase fills in the part of the primer opposite the overhang with sulfur-containing adenine analogs. HincII is added but only cuts the unmodified DNA strand. A DNA polymerase that lacks 5′ exonuclease activity enters at the site of the nick and begins to polymerize, displacing the initial primer strand downstream and building a new one which serves as more primer. SDA produces greater than 10⁷-fold amplification in 2 hours at 37° C. Unlike PCR and LCR, SDA does not require instrumented Temperature cycling.

Another method is a process for amplifying nucleic acid sequences from a DNA or RNA template which may be purified or may exist in a mixture of nucleic acids. The resulting nucleic acid sequences may be exact copies of the template, or may be modified. The process has advantages over PCR in that it increases the fidelity of copying a specific nucleic acid sequence, and it allows one to more efficiently detect a particular point mutation in a single assay. A target nucleic acid is amplified enzymatically while avoiding strand displacement. Three primers are used. A first primer is complementary to the first end of the target. A second primer is complementary to the second end of the target. A third primer which is similar to the first end of the target and which is substantially complementary to at least a portion of the first primer such that when the third primer is hybridized to the first primer, the position of the third primer complementary to the base at the 5′ end of the first primer contains a modification which substantially avoids strand displacement. This method is detailed in U.S. Pat. No. 5,593,840 to Bhatnagar et al. 1997, incorporated herein by reference.

Finally, recent application of DNA chips or microarray technology where DNA or oligonucleotides are immobilized on small solid support may also be used to rapidly sequence sample BRCA2 gene and analyze its expression. Typically, high density arrays of DNA fragment are fabricated on glass or nylon substrates by in situ light-directed combinatorial synthesis or by conventional synthesis followed by immobilization (Fodor et al. U.S. Pat. No. 5,445,934). Sample DNA or RNA may be amplified by PCR, labeled with a fluorescent tag, and hybridized to the microarray. Examples of this technology are provided in U.S. Pat. Nos. 5,510,270, 5,547,839, incorporated herein by reference.

All exonic and adjacent intronic sequences of the BRCA2 gene were obtained by end to end sequencing of five normal subjects in the manner described above followed by analysis of the data obtained. The data obtained provided us with the opportunity to establish the correct intronic/exonic structure of the BRCA2 gene. In addition, we evaluated six previously published normal polymorphisms (1342, 2457, 3199, 3624, 4035, and 7470) for correctness and frequency in the population, and to identify four additional polymorphisms not previously characterized (1093, 1593, 2908, and 9079).

### Gene Therapy

The polynucleotide(s) which result from either sense or antisense transcription of any exon or the entire coding sequence or fragments of BRCA2 gene may be used for gene therapy. A variety of methods are known for gene transfer, any of which might be available for use.

### Direct Injection of Recombinant DNA In Vivo

1. Direct injection of “naked” DNA directly with a syringe and needle into a specific tissue, infused through a vascular bed, or transferred through a catheter into endothelial cells.

2. Direct injection of DNA that is contained in artificially generated lipid vesicles or other encapsulating vehicles.

3. Direct injection of DNA conjugated to a target receptor structure, such as a diptheria toxin, an antibody or other suitable receptor.

4. Direct injection by particle bombardment. For example, the DNA may be coated onto gold particles and shot into the cells.

### Human Artificial Chromosomes

The gene delivery approach involves the use of human chromosomes that have been stripped down to contain only the essential components for replication and the genes desired for transfer.

### Receptor-Mediated Gene Transfer

DNA is linked to a targeting molecule that will bind to specific cell-surface receptors, inducing endocytosis and transfer of the DNA into mammalian cells. One such technique uses poly-L-lysine to link asialoglycoprotein to DNA. An adenovirus is also added to the complex to disrupt the lysosomes and thus allow the DNA to avoid degradation and move to the nucleus. Infusion of these particles intravenously has resulted in gene transfer into hepatocytes.

### Recombinant Virus Vectors

Several vectors may be used in gene therapy. Among them are the Moloney Murine Leukemia Virus (MoMLV) Vectors, the adenovirus vectors, the Adeno-Associated Virus (AAV) vectors, the herpes simplex virus (HSV) vectors, the poxvirus vectors, the retrovirus vectors, and human immunodeficiency virus (HIV) vectors.

### Gene Replacement and Repair

The ideal genetic manipulation for treatment of a genetic disease would be the actual replacement of the defective gene with a normal copy of the gene. Homologous recombination is the term used for switching out a section of DNA and replacing it with a new piece. By this technique, the defective gene may be replaced with a normal gene which expresses a functioning BRCA2 tumor growth inhibitor protein.

A complete description of gene therapy can also be found in “Gene Therapy A Primer For Physicians” 2d Ed. by Kenneth W. Culver, M. D. Publ. Mary Ann Liebert Inc. (1996). Two Gene Therapy Protocols for BRCA1 gene have been approved by the Recombinant DNA Advisory Committee for Jeffrey T. Holt et al. They are listed as 9602-148, and 9603-149 and are available from the NIH. Protocols for BRCA2 gene therapy may be similarly employed. The isolated BRCA2 gene may be synthesized or constructed from amplification products and inserted into a vector such as the LXSN vector.

### A BRCA2 Polypeptide or its Functional Equivalent

The growth of breast and ovarian cancer may be arrested or prevented by directly increasing the BRCA2 protein level where inadequate functional BRCA2 activity is responsible for breast and ovarian cancer. The cDNA and amino acid sequences of five novel BRCA2 haplotypes are disclosed herein (SEQ ID No:4-13). All or a fragment of BRCA2 protein may be used in therapeutic or prophylactic treatment of breast and ovarian cancer. Such a fragment may have a similar biological function as the native BRCA2 protein or may have a desired biological function as specified below. BRCA2 polypeptides or their functional equivalents including homologous and modified polypeptide sequences are also within the scope of the present invention. Changes in the native sequence may be advantageous in producing or using the BRCA2 derived polypeptides or functional equivalents suitable for therapeutic or prophylactic treatment of breast and ovarian cancer. For example, these changes may be desirable for producing resistance against in vivo proteolytic cleavage, for facilitating transportation and delivery of therapeutic reagents, for localizing and compartmentalizing tumor suppressing agents, or for expression, isolating and purifying the target species.

There are a variety of methods to produce an active BRCA2 polypeptide or a functional equivalent as a tumor growth inhibitor. For example, one or more amino acids may be substituted, deleted, or inserted using methods well known in the art (Maniatis et al., 1982). Considerations of polarity, charge, solubility, hydrophobicity, hydrophilicity and/or the amphiphathic nature of the amino acids play an important role in designing homologous polypeptide changes suitable for the intended treatment. In particular, conservative amino acid substitution using amino acids that are related in side-chain structure and charge may be employed to preserve the chemical and biological property. A homologous polypeptide typically contains at least 70% homology to the native sequence. Unnatural forms of the polypeptide may also be incorporated so long as the modification retains substantial biological activity. These unnatural polypeptides typically include structural mimics and chemical medications, which have similar three-dimensional structures as the active regions of the native BRCA2 protein. For example, these modifications may include terminal D-amino acids, cyclic peptides, unnatural amino acids side chains, pseudopeptide bonds, N-terminal acetylation, glycosylation, and biotinylation, etc. These unnatural forms of polypeptide may have a desired biological function, for example, they may be particularly robust in the presence of cellular or serum proteases and exopeptidase. An effective BRCA2 polypeptide or a functional equivalent may also be recognized by the reduction of the native BRCA2 protein. Regions of the BRCA2 protein may be systematically deleted to identify which regions are essential for tumor growth inhibitor activity. These smaller fragments of BRCA2 protein may then be subjected to structural and functional modification to derive therapeutically or prophylactically effective regiments. Finally, drugs, natural products or small molecules may be screened or synthesized to mimic the function of the BRCA2 protein. Typically, the active species retain the essential three-dimensional shape and chemical reactivity, and therefore retain the desired aspects of the biological activity of the native BRCA2 protein. The activity and function of BRCA2 may include transactivation, granin, DNA repair among others. Functions of BRCA2 protein are also reviewed in Bertwistle and Ashworth, Curr. Opin. Genet. Dev. 8(1): 14-20 (1998) and Zhang et al., Cell 92:433-436 (1998). It will be apparent to one skilled in the art that a BRCA2 polypeptide or a functional equivalent may be selected because such polypeptide or functional equivalent possesses similar biological activity as the native BRCA2 protein.

### Expression of the BRCA2 Protein and Polypeptide in Host Cells

All or fragments of the BRCA2 protein and polypeptide may be produced by host cells that are capable of directing the replication and the expression of foreign genes. Suitable host cells include prokaryotes, yeast cells, or higher eukaryotic cells, which contain an expression vector comprising all or a fragment of the BRCA2 cDNA sequence (SEQ. ID No: 4, 6, 8; 10, or 12) operatively linked to one or more regulatory sequences to produce the intended BRCA2 protein or polypeptide. Prokaryotes may include gram negative or gram positive organisms, for example E. coli or Bacillus strains. Suitable eukaryotic host cells may include yeast, virus, and mamalian systems. For example, Sf9 insect cells and human cell lines, such as COS, MCF7, HeLa, 293T, HBL100, SW480, and HCT116 cells.

A broad variety of suitable expression vectors are available in the art. An expression vector typically contains an origin of replication, a promoter, a phenotypic selection gene (antibiotic resistance or autotrophic requirement), and a DNA sequence coding for all or fragments of the BRCA2 protein. The expression vectors may also include other operatively linked regulatory DNA sequences known in the art, for example, stability leader sequences, secretory leader sequences, restriction enzyme cleavage sequences, polyadenylation sequences, and termination sequences, among others. The essential and regulatory elements of the expression vector must be compatible with the intended host cell. Suitable expression vectors containing the desired coding and control regions may be constructed using standard recombinant DNA techniques known in the art, many of which are described in Sambrook, et al., Molecular Cloning: A Laboratory Manual, Second Edition, Cold Spring Harbor Laboratory, Cold Spring Harbor, N.Y. (1989). For example, suitable origins of replication may include Col E1, SV40 viral and M13 origins of replication. Suitable promoters may be constitutive or inducible, for example, tac promoter, lac Z promoter, SV40 promoter, MMTV promoter, and LXSN promoter. Examples of selectable markers include neomycin, ampicillin, and hygromycin resistance and the like. Many suitable prokaryotic, viral and mammalian expression vectors may be obtained commercially, for example, from Invitrogen Corp., San Diego, Calif. or from Clontech, Palo Alto, Calif. It may be desirable that the BRCA2 protein or polypeptide is produced as a fusion protein to enhance the expression in selected host cells, to detect the expression in transfected cells, or to simplify the purification process. Suitable fusion partners for the BRCA2 protein or polypeptide are well known in the art and may include β-galactosidase, glutathione-S-transferase, and poly-histidine tag.

Expression vectors may be introduced into host cells by various methods known in the art. The transformation procedure used depends upon the host to be transformed. Methods for introduction of vectors into host cells may include calcium phosphate precipitation, electrosporation, dextran-mediated transfection, liposome encapsulation, nucleus microinjection, and viral or phage infection, among others.

Once an expression vector has been introduced into a suitable host cell, the host cell may be cultured under conditions permitting expression of large amounts of the BRCA2 protein or polypeptide. The expression product may be identified by many approaches well known in the art, for example, sequencing after PCR-based amplification, hybridization using probes complementary to the desired DNA sequence, the presence or absence of marker gene functions such as enzyme activity or antibiotic resistance, the level of mRNA production encoding the intended sequence, immunological detection of a gene product using monoclonal and polyclonal antibodies, such as Western blotting or ELISA. The BRCA2 protein or polypeptides produced in this manner may then be isolated following cell lysis and purified using various protein purification techniques known in the art, for example, ion exchange chromatography, gel filtration chromatography and immunoaffinity chromatography.

It is generally preferred that whenever possible, longer fragments of BRCA2 protein or polypeptide are used, particularly to include the desired functional domains of BRCA2 protein. Expression of shorter fragments of DNA may be useful in generating BRCA2 derived immunogen for the production of anti-BRCA2 antibodies. It should, of course, be understood that not all expression vectors, DNA regulatory sequences or host cells will function equally well to express the BRCA2 protein or polypeptides of the present invention. However, one of ordinary skill in the art may make a selection among expression vectors, DNA regulatory sequences, host cells, and codon usage in order to optimize expression using known technology in the art without undue experimentation. Studies of BRCA2 protein function and examples of genetic manipulation of BRCA2 protein are summarized in two recent review articles, Bertwistle and Ashworth, Curr. Opin. Genet. Dev. 8(1): 14-20 (1998) and Zhang et al., Cell 92:433-436 (1998).

### In Vitro Synthesis and Chemical Synthesis

Although it is preferred that fragments of the BRCA2 protein or polypeptides be obtained by overexpression in prokaryotic or eukaryotic host cells, the BRCA2 polypeptides or their functional equivalents may also be obtained by in vitro translation or synthetic means by methods known to those of ordinary skill in the art. For example, in vitro translation may employ an mRNA encoded by a DNA sequence coding for fragments of the BRCA2 protein or polypeptides. Chemical synthesis methodology such as solid phase synthesis may be used to synthesize a BRCA2 polypeptide structural mimic and chemically modified analogs thereof. The polypeptides or the modifications and mimic thereof produced in this manner may then be isolated and purified using various purification techniques, such as chromatographic procedures including ion exchange chromatography, gel filtration chromatography and immunoaffinity chromatography.

### Protein Replacement Therapy

The tumor suppressing function of BRCA2 suggests that various BRCA2 protein targeted therapies may be utilized in treating and preventing tumors in breast and ovarian cancer. The present invention therefore includes therapeutic and prophylactic treatment of breast and ovarian cancer using therapeutic pharmaceutical compositions containing the BRCA2 protein, polypeptides, or their functional equivalents. For example, protein replacement therapy may involve directly administering the BRCA2 protein, a BRCA2 polypeptide, or a functional equivalent in a pharmaceutically effective carrier. Alternatively, protein replacement therapy may utilize tumor antigen specific antibody fused to fragments of the BRCA2 protein, a polypeptide, or a functional equivalent to deliver anti-cancer regiments specifically to the tumor cells.

To prepare the pharmaceutical compositions of the present invention, an active BRCA2 protein, a BRCA2 polypeptide, or its functional equivalent is combined with a pharmaceutical carrier selected and prepared according to conventional pharmaceutical compounding techniques. A suitable amount of the composition may be administered locally to the site of a tumor or systemically to arrest the proliferation of tumor cells. The methods for administration, may include parenteral, oral, or intravenous, among others according to established protocols in the art.

Pharmaceutically acceptable solid or liquid carriers or components which may be added to enhance or stabilize the composition, or to facilitate preparation of the composition include, without limitation, syrup, water, isotonic solution, 5% glucose in water or buffered sodium or ammonium acetate solution, oils, glycerin, alcohols, flavoring agents, preservatives, coloring agents, starches, sugars, diluents, granulating agents, lubricants, binders, and sustained release materials. The dosage at which the therapeutic compositions are administered may vary within a wide range and depends on various factors, such as the stage of cancer progression, the age and condition of the patient, and may be individually adjusted.

### Diagnostic Reagents

The BRCA2 protein, polypeptides, their functional equivalents, antibodies, and polynucleotides may be used in a wide variety of ways in addition to gene therapy and protein replacement therapy. They may be useful as diagnostic reagents to measure normal or abnormal activity of BRCA2 at the DNA, RNA, and protein level. The present invention therefore encompasses the diagnostic reagents derived from the BRCA2 cDNA and protein sequences as set forth in SEQ. ID. Nos: 4-13. These reagents may be utilized in methods for monitoring disease progression, for determining patients suited for gene and protein replacement therapy, or for detecting the presence or quantifying the amount of a tumor growth inhibitor following such therapy. Such methods may involve conventional histochemical techniques, such as obtaining a tumor tissue from the patient, preparing an extract and testing this extract for tumor growth or metabolism. For example, the test for tumor growth may involve measuring abnormal BRCA2 activity using conventional diagnostic assays, such as Southern, Northern, and Western blotting, PCR, RT-PCR, and immunoprecipitation. In biopsies of tumor tissues, the loss of BRCA2 expression in tumor tissue may be verified by RT-PCR and Northern blotting at the RNA level. A Southern blot analysis, genomic PCR, or fluorescence in situ hybridization (FISH) may also be performed to examine the mutations of BRCA2 at the DNA level. And, a Western blotting, protein truncation assay, or immunoprecipitation may be utilized to analysis the effect at the protein level.

These diagnostic reagents are typically either covalently or non convalently attached to a detectable label. Such a label includes a radioactive label, a calorimetric enzyme label, a fluorescence label, or an epitope label. Frequently, a reporter gene downstream of the regulatory sequences is fused with the BRCA2 protein or polypeptide to facilitate the detection and purification of the target species. Commonly used reporter genes in BRCA2 fusion proteins include β-galactosidase and luciferase gene.

The BRCA2 protein, polypeptides, their functional equivalents, antibodies, and polynucleotides may also be useful in the study of the characteristics of BRCA2 proteins, such as structure and function of BRCA2 in oncogenesis or subcellular localization of BRCA2 protein in normal and cancerous cell. For example, yeast two-hybrid system has been used in the study of cellular function of BRCA2 to identify the regulator and effector of BRCA2 tumor suppressing function (Sharan et al., Nature 386:804-810 (1997) and Katagiri et al., Genes, Chromosomes & Cancer 21:217-222 (1988)). In addition, the BRCA2 protein, polypeptides, their functional equivalents, antibodies, and polynucleotides may also be used in in vivo cell based and in vitro cell free assays to screen natural products and synthetic compounds which may mimic, regulate or stimulate BRCA2 protein function.

### Antisense Inhibition

Antisense suppression of endogenous BRCA2 expression may assess the effect of BRCA2 protein on cell growth inhibition using known method in the art (Crooke, Annu. Rev. Pharmacol. Toxicol. 32:329-376 (1992) and Robinson-Benion and Holt, Methods Enzymol. 254.363-375 (1995)). Given the cDNA sequence as set forth in SEQ ID. NO: 4, 6, 8, 10, and 12, one of skill in the art can readily obtain anti-sense strand of DNA and RNA sequences to interfere with the production of wild-type BRCA2 protein or the mutated form of BRCA2 protein. Alternatively, antisense oligonucleotide may be designed to target the control sequences of BRCA2 gene to reduce or prevent the expression of the endogenous BRCA2 gene.

### Antibodies

The BRCA2 protein, polypeptides, or their functional equivalents may be used as immunogens to prepare polyclonal or monoclonal antibodies capable of binding the BRCA2 derived antigens in a known manner (Harlow & Lane, Antibodies: A Laboratory Manual, Cold Spring Harbor Laboratory, Cold Spring Harbor, N.Y., 1988). These antibodies may be used for the detection of the BRCA2 protein, polypeptides, or a functional equivalent in an immunoassay, such as ELISA, Western blot, radioimmunoassay, enzyme immunoassay, and immunocytochemistry. Typically, an anti-BRCA2 antibody is in solution or is attached to a solid surface such as a plate, a particle, a bead, or a tube. The antibody is allowed to contact a biological sample or a blot suspected of containing the BRCA2 protein or polypeptide to form a primary immunocomplex. After sufficient incubation period, the primary immunocomplex is washed to remove any non-specifically bound species. The amount of specifically bound BRCA2 protein or polypeptide may be determined using the detection of an attached label or a marker, such as a radioactive, a fluorescent, or an enzymatic label. Alternatively, the detection of BRCA2 derived antigen is allowed by forming a secondary immunocomplex using a second antibody which is attached with a such label or marker. The antibodies may also be used in affinity chromatography for isolating or purifying the BRCA2 protein, polypeptides or their functional equivalents.

### Example 1

### Determination of the Coding Sequence Haplotypes of the BRCA2 Gene from Normal Individuals

Approximately 150 volunteers were screened in order to identify individuals with no cancer history in their immediate family (i.e. first and second degree relatives). Each person was asked to fill out a hereditary cancer prescreening questionnaire (See TABLE I). Five of these were randomly chosen for end-to-end sequencing of their BRCA2 gene. A first degree relative is a parent, sibling, or offspring. A second degree relative is an aunt, uncle, grandparent, grandchild, niece, nephew, or half-sibling.

Genomic DNA was isolated from white blood cells of five normal subjects selected from analysis of their answers to the questions above. Dideoxy sequence analysis was performed following polymerase chain reaction amplification.

All exons of the BRCA2 gene were subjected to direct dideoxy sequence analysis by asymmetric amplification using the polymerase chain reaction (PCR) to generate a single stranded product amplified from this DNA sample. Shuldiner, et al., Handbook of Techniques in Endocrine Research, p. 457-486, DePablo, F., Scanes, C., eds., Academic Press, Inc., 1993. Fluorescent dye was attached for automated sequencing using the Taq Dye Terminator Kit (Perkin-Elmer® cat #401628). DNA sequencing was performed in both forward and reverse directions on an Applied Biosystems, Inc. (ABI) automated sequencer (Model 377). The software used for analysis of the resulting data was “Sequence Navigator” purchased through ABI.

### 1. Polymerase Chain Reaction (PCR) Amplification

Genomic DNA (100 nanograms) extracted from white blood cells of five normal subjects. Each of the five samples was sequenced end to end. Each sample was amplified in a final volume of 25 microliters containing 1 microliter (100 nanograms) genomic DNA, 2.5 microliters 10×PCR buffer (100 mM Tris, pH 8.3, 500 mM KCl, 1.2 mM MgCl₂), 2.5 microliters 10×dNTP mix (2 mM each nucleotide), 2.5 microliters forward primer, 2.5 microliters reverse primer, and 1 microliter Taq polymerase (5 units), and 13 microliters of water.

The primers in TABLE II below were used to carry out amplification of the various sections of the BRCA2 gene samples. The primers were synthesized on an DNA/RNA Synthesizer Model 394®.

Thirty-five cycles were performed, each consisting of denaturing (95° C.; 30 seconds), annealing (55° C.; 1 minute), and extension (72° C.; 90 seconds), except during the first cycle in which the denaturing time was increased to 5 minutes, and during the last cycle in which the extension time was increased to 5 minutes.

PCR products were purified using Qia-Quick® PCR purification kits (Qiagen®, cat #28104; Chatsworth, Calif.). Yield and purity of the PCR product are determined spectrophotometrically at OD₂₆₀ on a Beckman DU 650 spectrophotometer.

### 2. Dideoxy Sequence Analysis

Fluorescent dye was attached to PCR products for automated sequencing using the Taq Dye Terminator Kit (Perkin-Elmer® cat #401628). DNA sequencing was performed in both forward and reverse directions on an Applied Biosystems, Inc. (ABI) Foster City, Calif., automated sequencer (Model 377). The software used for analysis of the resulting data was “Sequence Navigator®” purchased through ABI.

### 3. Results

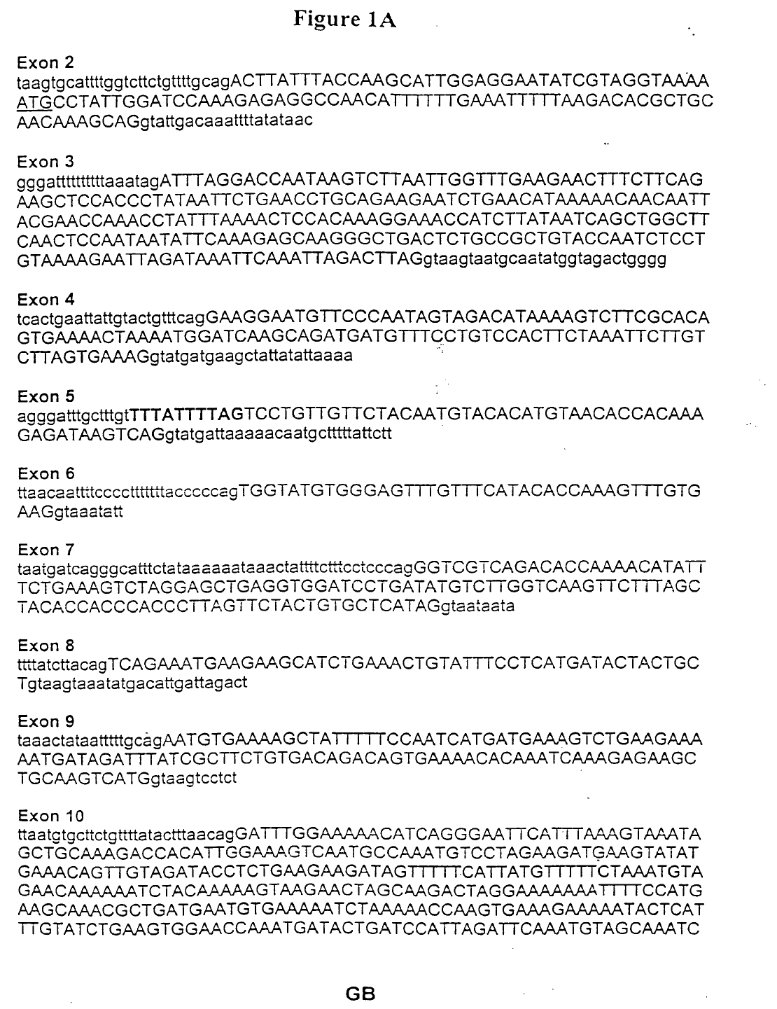

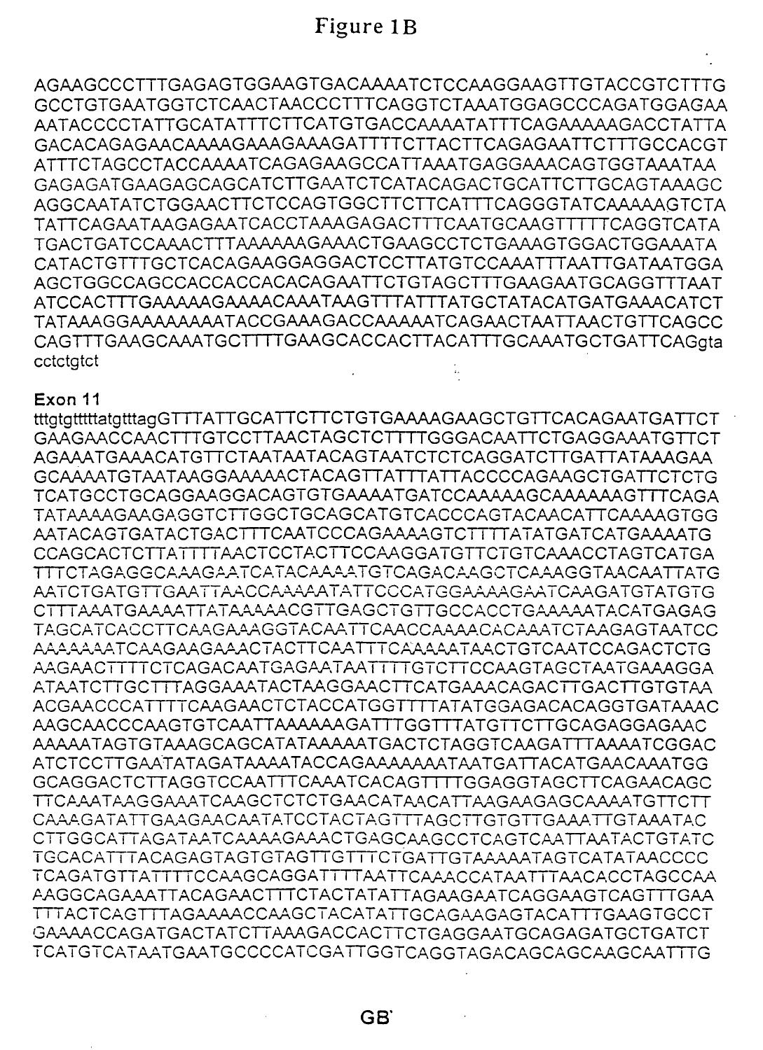

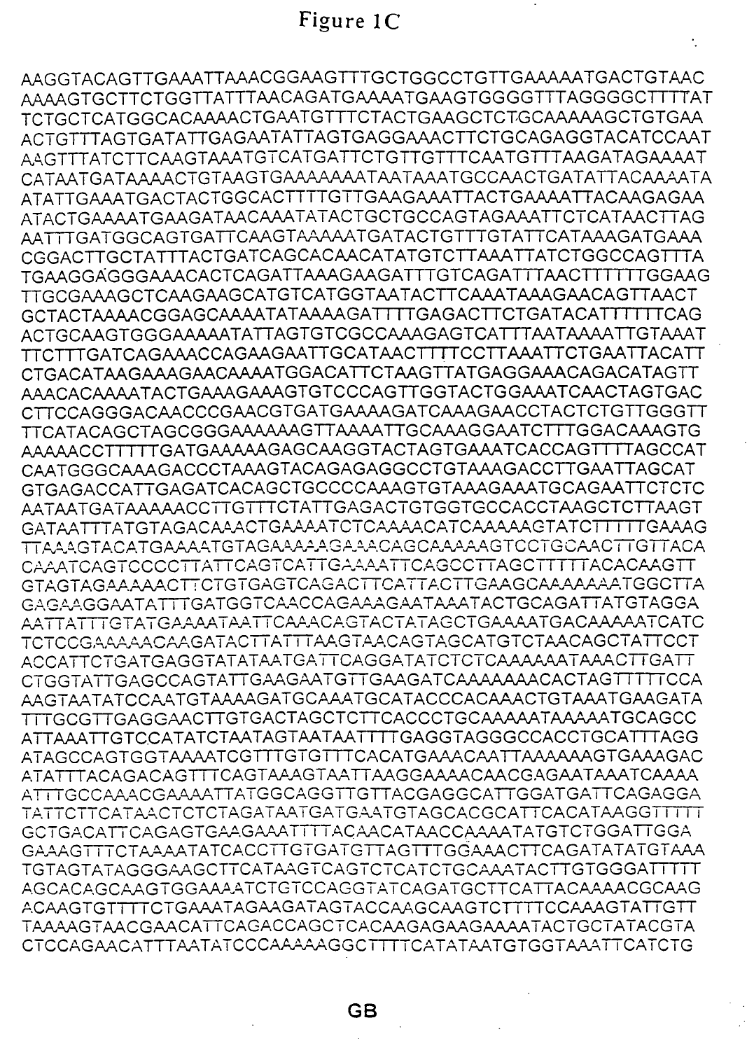

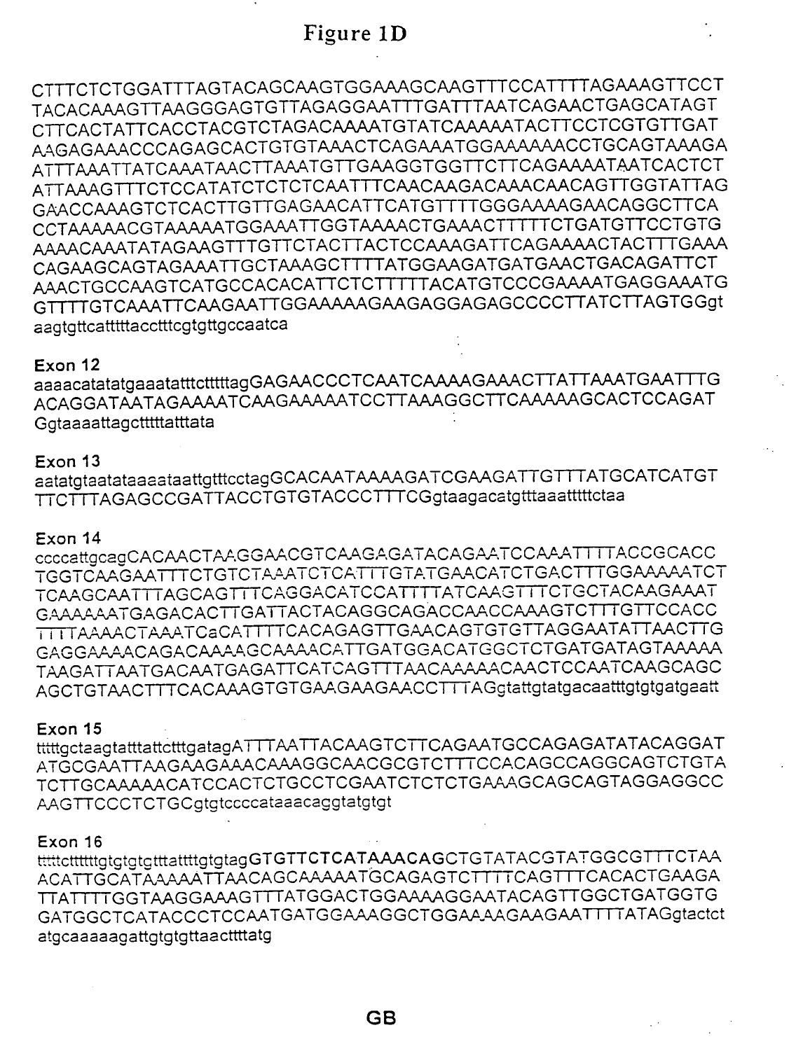

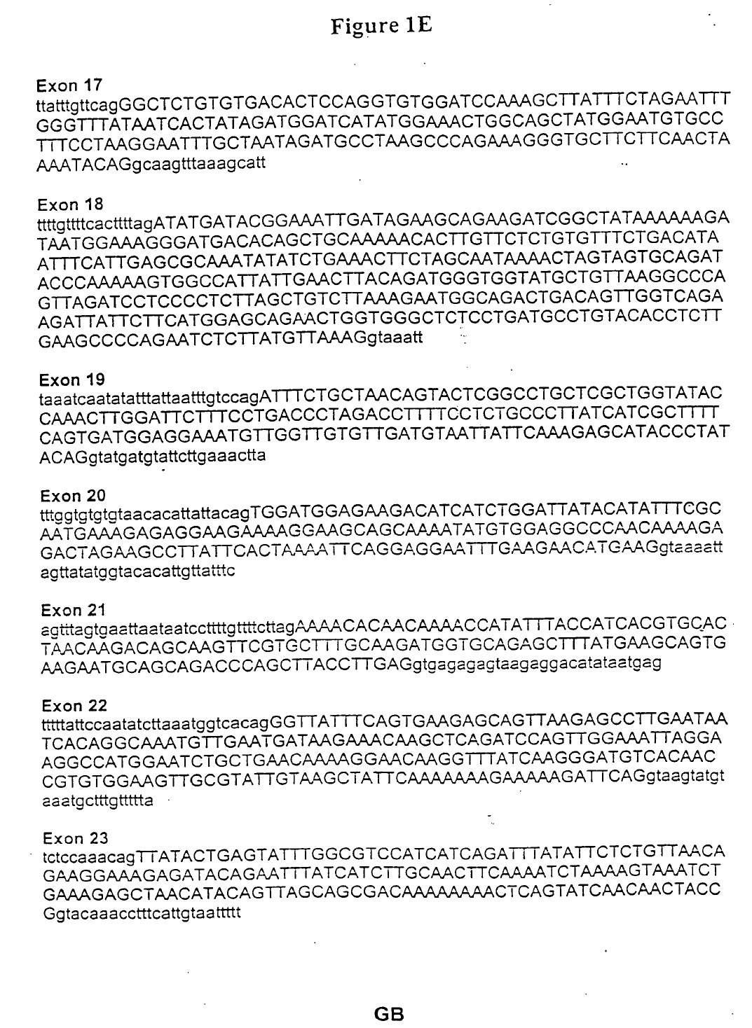

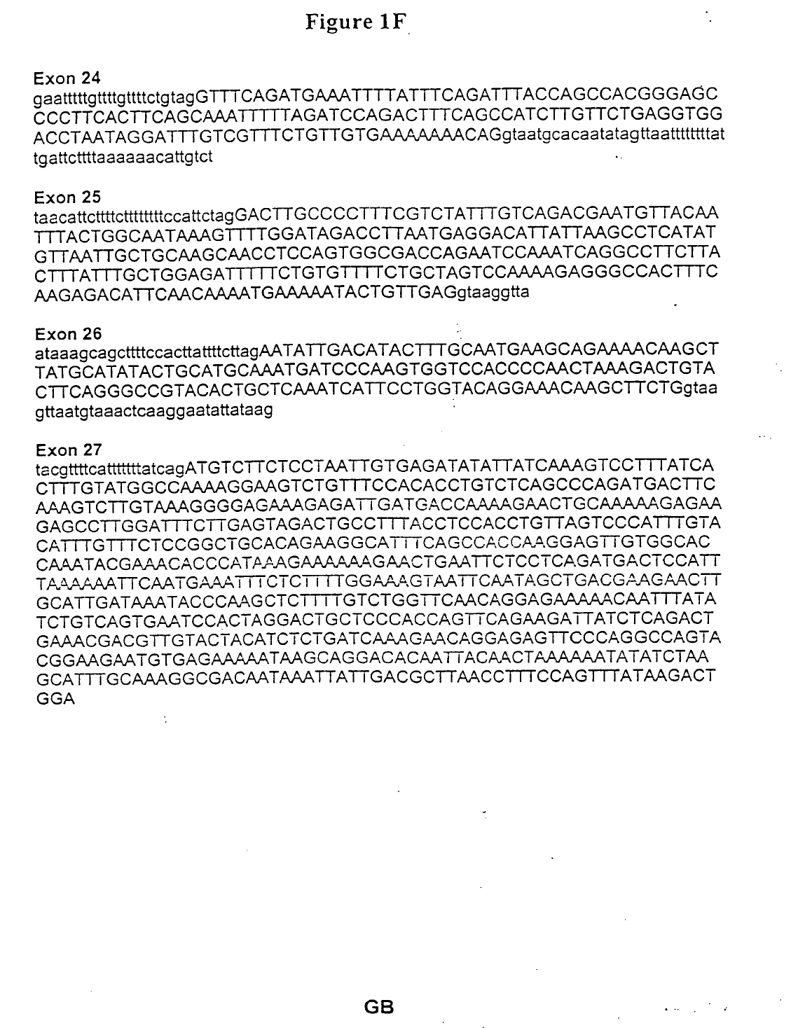

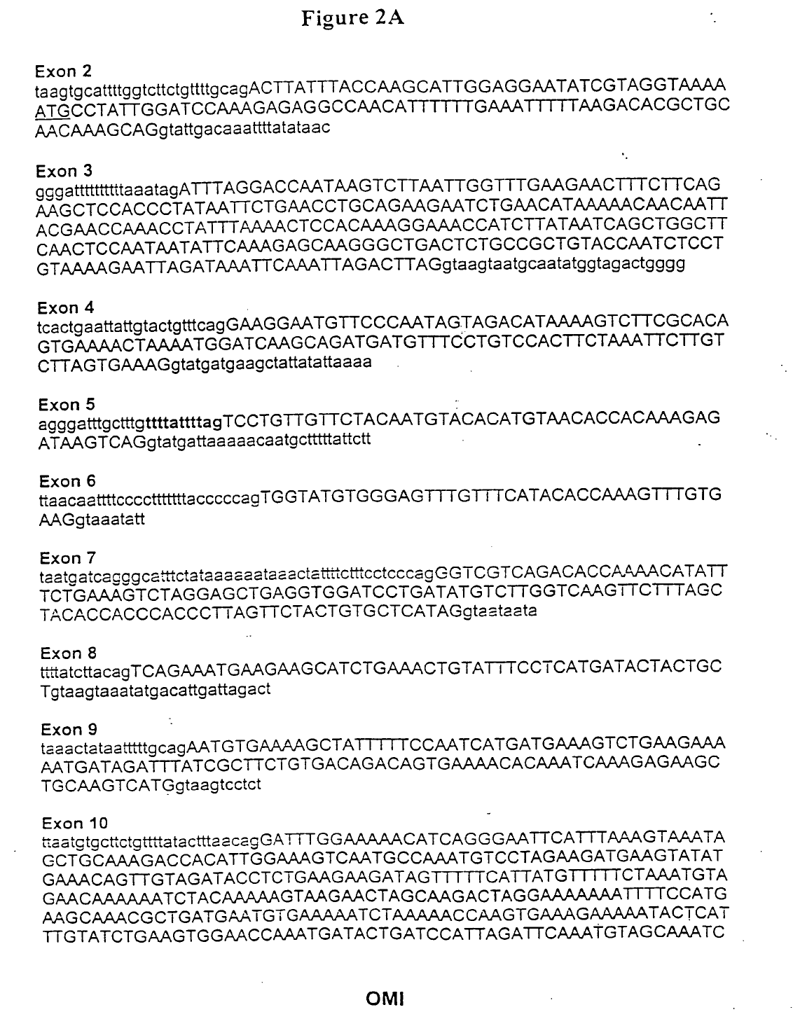

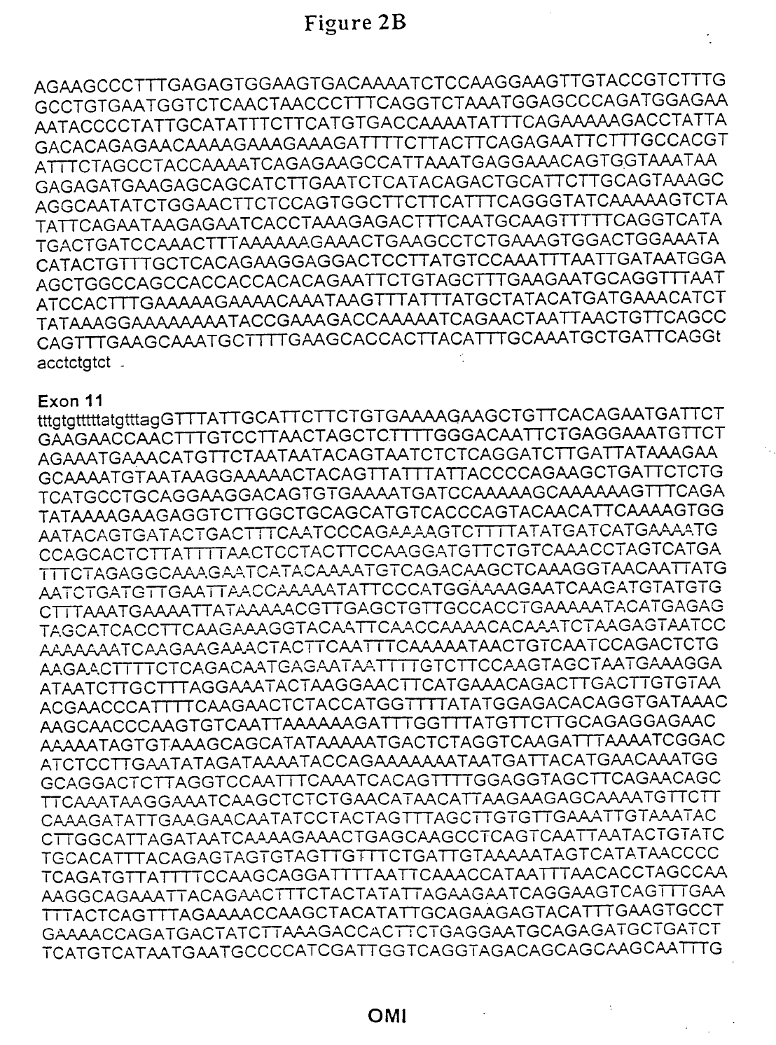

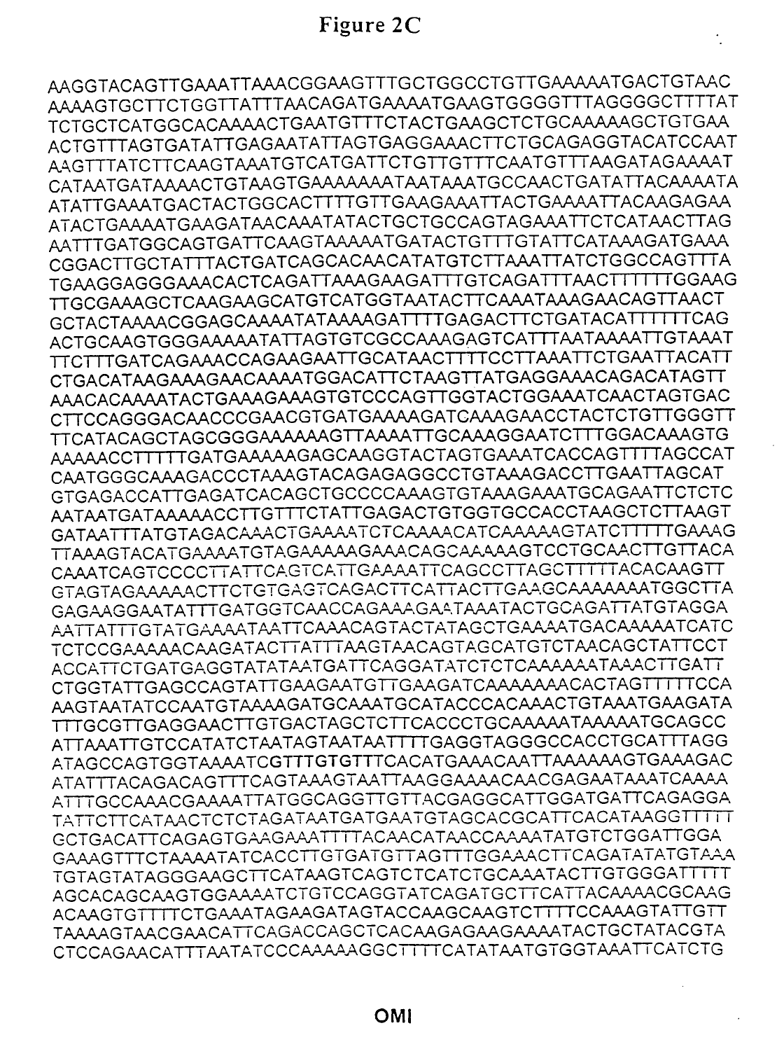

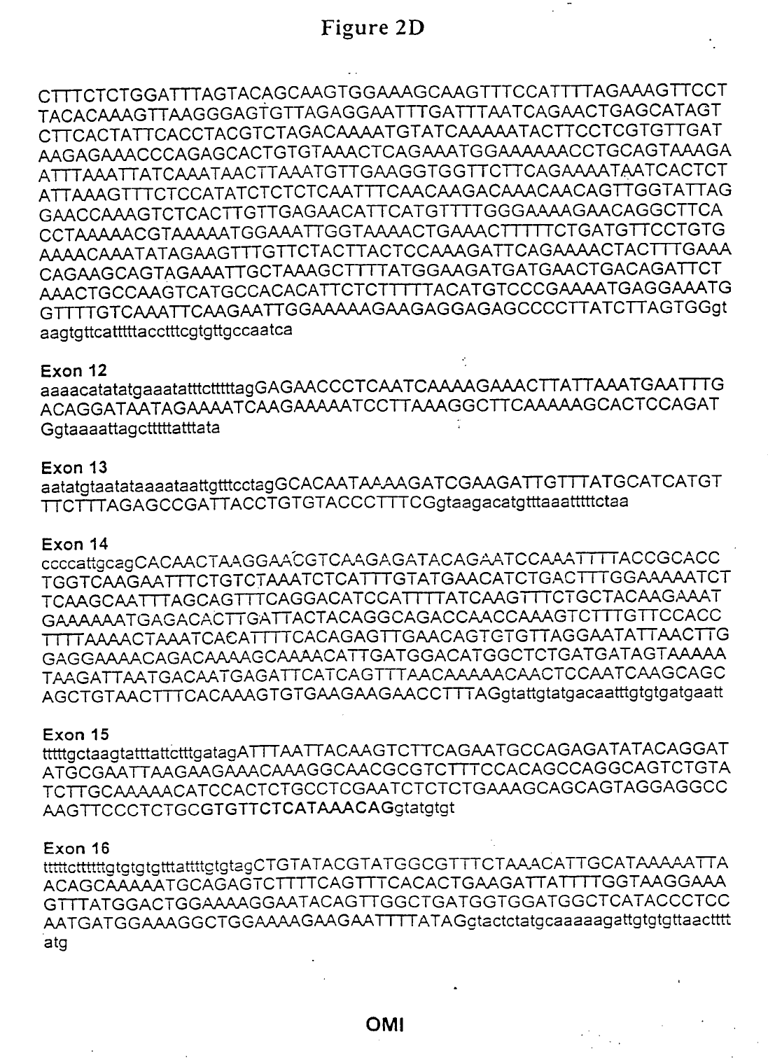

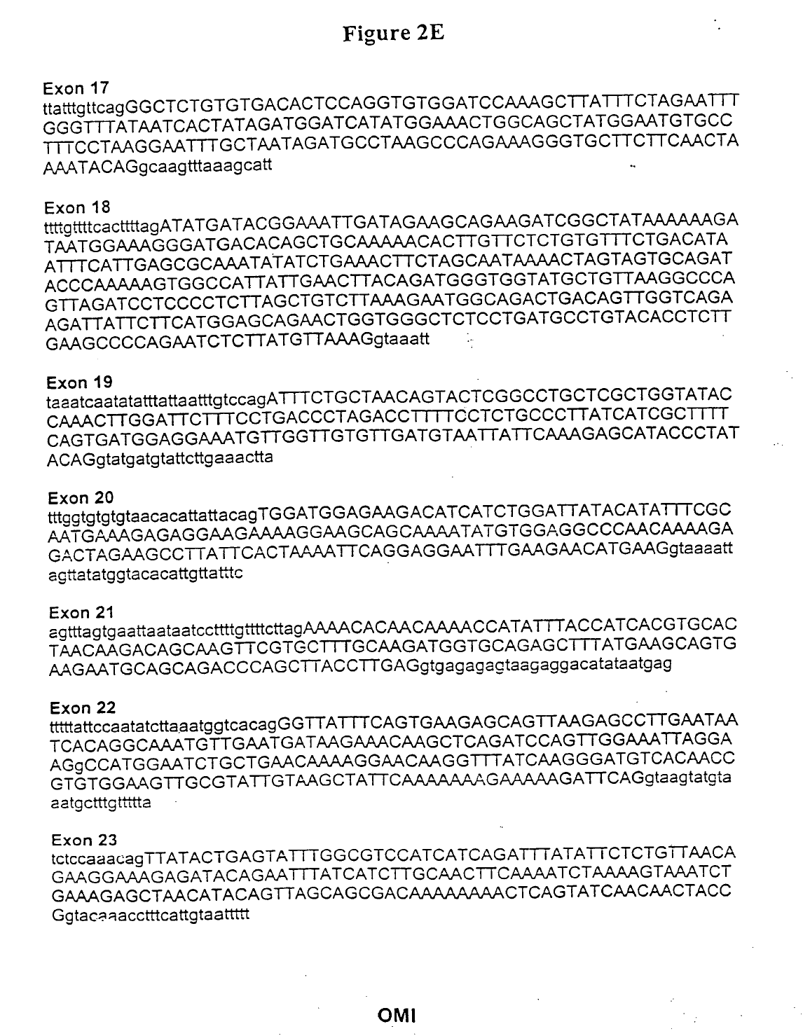

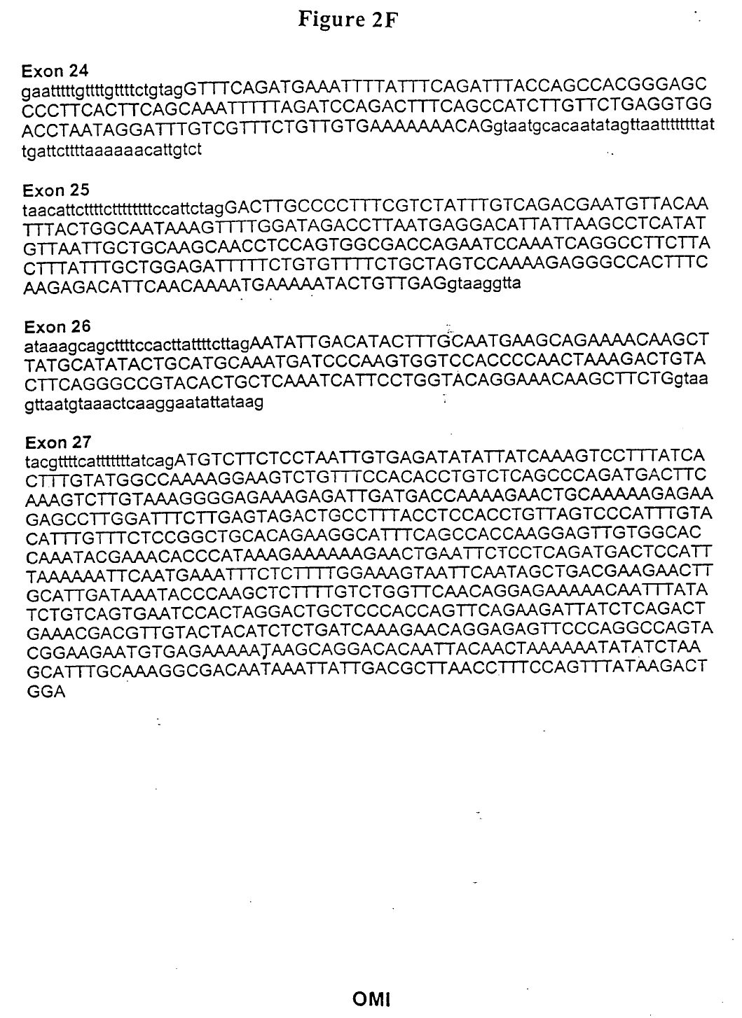

Based upon the sequencing of the five normal individuals, it was determined that the standard sequence found in both GenBank and BIC were inaccurate. In Genbank, a 10 bp stretch (5′-TTTATTTTAG-3′) was mistakenly listed as exonic at the 5′ end of exon 5 while it should be intronic which would not be included in the cDNA and resultant protein. In addition, a more detrimental error that has the significant potential to lead to an incorrect diagnosis of breast cancer propensity exists in both Genbank and BIC: a sequence of 16 bp (5′-GTGTTCTCATAAACAG-3′) should be at the end of exon 15, but instead is listed at the beginning of exon 16 in the database. The disclosure and listing of GenBank is shown in FIG. 1. The correct intron/exon sequence of BRCA2 is presented in FIG. 2, wherein,
(1) a 10 bp stretch (5′-TTTATTTTAG-3′) is intronic at 3′ end of intron 4, rather than at the 5′ end of exon 5 (corrected exon 5 is listed as SEQ. ID. NO: 1) and
(2) a 16 bp stretch (5′-GTGTTCTCATAAACAG-3′) is exonic at the 3′ end of exon 15, rather than at the 5′ end of exon 16 (corrected exons 15 and 16 are listed as SEQ. ID. No: 2 and 3 respectively)

The BIC BRCA2 sequence also contains sequence errors in which a stretch of nine nucleotides at positions 5554-5460 is listed as CGTTTGTGT (amino acids: Arg-Leu-Cys). The correct sequence at these positions is GTTTGTGTT (amino acids: Val-Cys-Val). In addition, the BIC BRCA2 nucleotides at positions 2024 (codon 599), 4553 (codon 1442), 4815 (codon 1529), 5841 (codon 1871), and 5972 (codon 1915) are T, T, A, C, and T respectively, wherein the correct nucleotides at these positions are C, C, G, T, and C respectively. Among them, the nucleotide errors at codon 599, 1442, 1915 result in amino acids changes.

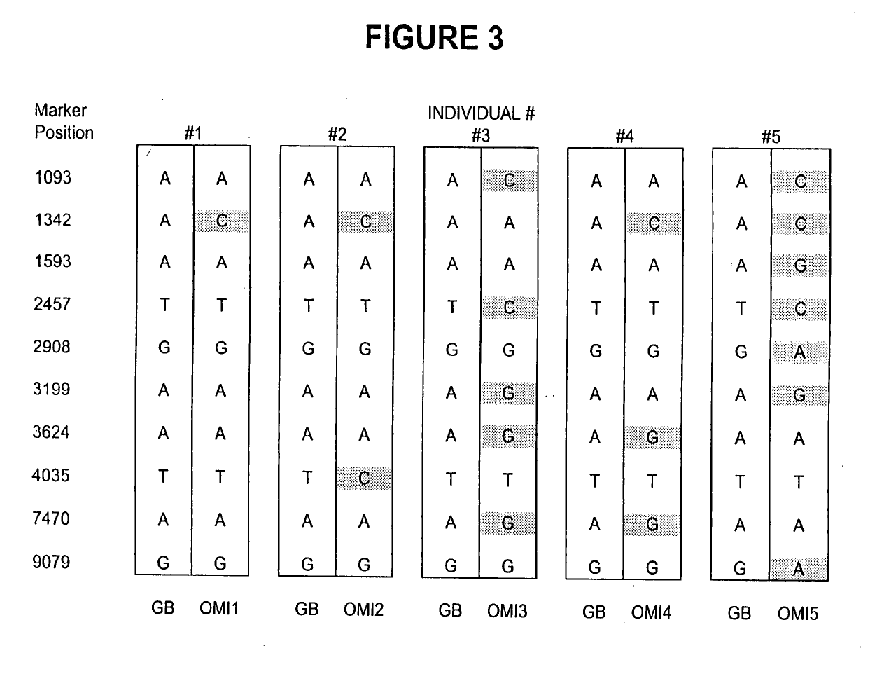

Additional differences in the nucleic acids of the five normal individuals were found in ten polymorphic locations. The changes and their positions are found in TABLE III. The individual haplotypes of each chromosome of BRCA2 are displayed in FIG. 3. In each case, the initial haplotype reported in Genbank (accession number U43746) was subtracted to determine the new haplotypes OMI 1-5. Thus, the Genbank sequence only represents 50% of the haplotypes found; the five new BRCA2^{(omi 1-5)} DNA sequences are shown as SEQ. ID. NO: 4, 6, 8, 10, and 12, respectively (See FIG. 3), and the corresponding polypeptides are listed as SEQ. ID. NO: 5, 7, 9, 11, and 13 respectively. In combination, these seven haplotypes represent a functional allele profile for the BRCA2 gene.

The data show that for each of the samples, all exons of BRCA2 were identical except in the region of ten polymorphisms. Six of these polymorphisms were previously identified (Tartigan et al., Nature Genetics 12: 333-337 (1996); Phelan et al., Nature Genetics 13: 120-122 (1996); Couch et al., Nature Genetics 13: 123-125 (1996); Teng, et al., Nature Genetics 13: 241-244 (1996); Schubert et al 60: 1031-1040 (1997)), but four were unique to this work. Even though the individual polymorphisms may have been identified, none of these complete haplotypes has been previously determined.

TABLE I
Hereditary Cancer Pre-Screening Questionnaire

Part A: Answer the following questions about your family

| | |
|---|---|
| 1. | To your knowledge, has anyone in your family been diagnosed with a very specific hereditary |
| | colon disease called Familial Adenomatous Polyposis (FAP)? |
| 2. | To your knowledge, have you or any aunt had breast cancer diagnosed before the age 35? |
| 3. | Have you had Inflammatory Bowel Disease, also called Crohn's Disease or Ulcerative Colitis, |
| | for more than 7 years? |

Part B: Refer to the list of cancers below for your responses only to questions in Part B

| | | |
|---|---|---|
| Bladder Cancer | Lung Cancer | Pancreatic Cancer |
| Breast Cancer | Gastric Cancer | Prostate Cancer |
| Colon Cancer | Malignant Melanoma | Renal Cancer |
| Endometrial Cancer | Ovarian Cancer | Thyroid Cancer |

| | |
|---|---|
| 4. | Have your mother or father, your sisters or brothers or your children had any of the listed |
| | cancers? |
| 5 | Have there been diagnosed in your mother's brothers or sisters, or your mother's parents |
| | more than one of the cancers in the above list? |
| 6. | Have there been diagnosed in your father's brothers or sisters, or your father's parents more |
| | than one of the cancers in the above list? |

Part C: Refer to the list of relatives below for responses only to questions in Part C

| | |
|---|---|
| You | Your mother |
| Your sisters or brothers | Your mother's sisters or brothers (maternal aunts & |
| | uncles) |
| Your children | Your mother's parents (maternal grandparents) |

| | |
|---|---|
| 7. | Have there been diagnosed in these relatives 2 or more identical types of cancer? Do not |
| | count “simple” skin cancer, also called basal cell or squamous cell skin cancer. |
| 8. | Is there a total of 4 or more of any cancers in the list of relatives above other than |
| | “simple” skin cancers? |

Part D: Refer to the list of relatives below for responses only to questions in Part D.

| | |
|---|---|
| You | Your father |
| Your sisters or brothers | Your father's sisters or brothers (paternal aunts and |
| | uncles) |
| Your children | Your father's parents (paternal grandparents) |

| | |
|---|---|
| 9. | Have there been diagnosed in these relatives 2 or more identical types of cancer? Do not |
| | count “simple” skin cancer, also called basal cell or squamous cell skin cancer. |
| 10. | Is there a total of 4 or more of any cancers in the list of relatives above other than “simple” |
| | skin cancers? |

© Copyright 1996, OncorMed, Inc.

TABLE II
BRCA2 PRIMER SEQUENCES

| | | | | | |
|---|---|---|---|---|---|
| | | SEQUENCE (5′ TO 3′) NOTE: | | | |
| | | M13 TAIL INCLUDED | | PCR | SEQ. |
| | | M13 FORWARD = TGT AAA ACG ACG GCC AGT | Oligo | Product | ID. |
| Exon | Label | M13 REVERSE = CAG GAA ACA GCT ATG ACC | Length | Length | Number |

| | | | | | |
|---|---|---|---|---|---|
| 2 | BRCA2-2F | 5′-TGA GTT TTA CCT CAG TCA CA-3′ | 20 | 263 | 14 |
| 2 | BRCA2-2R/M 13R | 5′-CAG GAA ACA GCT ATG ACC CTG TGA CGT ACT GGG TTT TTA | 41 | | 15 |
| | | GC-3′ | | | |
| 3 | BRCA2-3FII | 5′-GAT CTT TAA CTG TTC TGG GTC ACA-3′ | 24 | 364 | 16 |
| 3 | BRCA2-3RII | 5′-CCC AGC ATG ACA CAA TTA ATG A-3′ | 22 | | 17 |
| 4 | BRCA2-4F/M 13F | 5′-TGT AAA ACG ACG GCC AGT AGA ATG CAA ATT TAT AAT CCA | 44 | 268 | 18 |
| | | GAG TA-3′ | | | |
| 4 | BRCA2-4R-1A | 5′-ATC AGA TTC ATC TTT ATA GAA C-3′ | 22 | | 19 |
| 5 & 6 | BRCA2-5 + 6F/M13F | 5′-TGT AAA ACG ACG GCC AGT TGT GTT GGC ATT TTA AAC ATC | 40 | 453 | 20 |
| | | A-3′ | | | |
| 5 & 6 | BRCA2-5 + 6R/M13R | 5′-CAG GAA ACA GCT ATG ACC CAG GGC AAA GGT ATA ACG CT-3′ | 38 | | 21 |
| 7 | BRCA2-7F/M13F | 5′-TGT AAA ACG ACG GCC AGT TAA GTG AAA TAA AGA GTG AA-3′ | 38 | 248 | 22 |
| 7 | BRCA2-7R/M13R | 5′-CAG GAA ACA GCT ATG ACC AGA AGT ATT AGA GAT GAC-3′ | 36 | | 23 |
| 8 | BRCA2-8F/M13F | 5′-TGT AAA ACG ACG GCC AGT GCC ATA TCT TAC CAC CTT GTG | 40 | 319 | 24 |
| | | A-3′ | | | |
| | BRCA2-8FIA | 5′-TTG CAT TCT AGT GAT AAT ATA C-3′ | 22 | 143 | 25 |
| 8 | BRCA2-8RIA | 5′-AAT TGT TAG CAA TTT CAA C-3′ | 19 | | 26 |
| 9 | BRCA2-9F/M13F | 5′-TGT AAA ACG ACG GCC AGT TGG ACC TAG GTT GAT TGC AGA | 40 | 338 | 27 |
| | | T-3′ | | | |
| 9 | BRCA2-9R/M13R | 5′-CAG GAA ACA GCT ATG ACC TAA ACT GAG ATC ACG GGT GAC | 40 | | 28 |
| | | A-3′ | | | |
| 10A | BRCA2-10AF | 5′-GAA TAA TAT AAA TTA TAT GGC TTA-3′ | 24 | 255 | 29 |
| 10A | BRCA2-10AR/M13R | 5′-CAG GAA ACA GCT ATG ACC CCT AGT CTT GCT AGT TCT T-3′ | 37 | | 30 |
| 10B | BRCA2-10BF/M13F | 5′-TGT AAA ACG ACG GCC AGT ARC TGA AGT GGA ACC AAA TGA | 42 | 621 | 31 |
| | | TAC-3′ | | | |
| 10B | BRCA2-10BR/M13R | 5′-CAG GAA ACA GCT ATG ACC ACG TGG CAA AGA ATT CTC TGA | 44 | | 32 |
| | | AGT AA-3′ | | | |
| 10C | BRCA2-10CF/M13F | 5′-TGT AAA ACG ACG GCC AGT CAG CAT CTT GAA TCT CAT ACA | 40 | 508 | 33 |
| | | G-3′ | | | |
| 10C | BRCA2-10CRII | 5′-AGA CAG AGG TAC CTG AAT C-3′ | 19 | | 34 |
| 11 | BRCA2-11AF-M13 | 5′-TGT AAA ACG ACG GCC AGT TGG TAC TTT AAT TTT GTC ACT | 40 | 304 | 35 |
| | | T-3′ | | | |
| 11 | BRCA2-11AR-M13 | 5′-CAG GAA ACA GCT ATG ACC TGC AGG CAT GAC AGA GAA T-3′ | 37 | | 36 |
| 11 | BRCA2-11BF | 5′-AAG AAG CAA AAT GTA ATA AGG A-3′ | 22 | 411 | 37 |
| 11 | BRCA2-11BR | 5′-CAT TTA AAG CAC ATA CAT CTT G-3′ | 22 | | 38 |
| 11 | BRCA2-11CF | 5′-TCT AGA GGC AAA GAA TCA TAC-3′ | 21 | 349 | 39 |
| 11 | BRCA2-11CR | 5′-CAA GAT TAT TCC TTT CAT TAG C-3′ | 22 | | 40 |
| 11 | BRCA2-11DF | 5′-AAC CAA AAC ACA AAT CTA AGA G-3′ | 22 | 344 | 41 |
| 11 | BRCA2-11DR | 5′-GTC ATT TTT ATA TGC TGC TTT AC-3′ | 23 | | 42 |
| 11 | BRCA2-11EF | 5′-GGT TTT ATA TGG AGA CAC AGG-3′ | 21 | 369 | 43 |
| 11 | BRCA2-11ER | 5′-GTA TTT ACA ATT TCA ACA CAA GC-3′ | 23 | | 44 |
| 11 | BRCA2-11FF | 5′-ATC ACA GTT TTG GAG GTA GC-3′ | 20 | 368 | 45 |
| 11 | BRCA2-11FR | 5′-CTG ACT TCC TGA TTC TTC TAA-3′ | 21 | | 46 |
| 11 | BRCA2-11GF | 5′-CTC AGA TGT TAT TTT CCA AGC-3′ | 21 | 366 | 47 |
| 11 | BRCA2-11GR | 5′-CTG TTA AAT AAC CAG AAG CAC-3′ | 21 | | 48 |
| 11 | BRCA2-11HF | 5′-AGG TAG ACA GCA GCA AGC-3′ | 18 | 360 | 49 |
| 11 | BRCA2-11HR | 5′-GTA ATA TCA GTT GGC ATT TAT T-3′ | 22 | | 50 |
| 11 | BRCA2-11IF | 5′-TGC AGA GGT ACA TCC AAT AAG-3′ | 21 | 326 | 51 |
| 11 | BRCA2-11IR | 5′-GAT CAG TAA ATA GCA AGT CCG-3′ | 21 | | 52 |
| 11 | BRCA2-11JF | 5′-TAC TGA AAA TGA AGA TAA CAA AT-3′ | 23 | 477 | 53 |
| 11 | BRCA2-11JR | 5′-ATT TTG TTC TTT CTT ATG TCA G-3 | 22 | | 54 |
| 11 | BRCA2-11KF-M13 | 5′-TGT AAA ACG ACG GCC AGT CTA CTA AAA CGG AGC AA-3′ | 35 | 382 | 55 |
| 11 | BRCA2-11KR-M13 | 5′-CAG GAA ACA GCT ATG ACC GTA TGA AAA CCC AAC AG-3′ | 35 | | 56 |
| 11 | BRCA2-11LF | 5′-CAC AAA ATA CTG AAA GAA AGT G-3′ | 22 | 374 | 57 |
| 11 | BRCA2-11LR | 5′-GGC ACC ACA GTC TCA ATA G-3′ | 19 | | 58 |
| 11 | BRCA2-11MF | 5′-GCA AAG ACC CTA AAG TAC AG-3′ | 20 | 409 | 59 |
| 11 | BRCA2-11MR | 5′-CAT CAA ATA TTC CTT CTC TAA G-3′ | 22 | | 60 |
| 11 | BRCA2-11NF-M13 | 5′-TGT AAA ACG ACG GCC AGT GAA AAT TCA GCC TTA GC-3′ | 35 | 306 | 61 |
| 11 | BRCA2-11NR-M13 | 5′-CAG GAA ACA GCT ATG ACC ATC AGA ATG GTA GGA AT-3′ | 35 | | 62 |
| 11 | BRCA2-11OF | 5′-GTA CTA TAG CTG AAA ATG ACA A-3′ | 22 | 383 | 63 |
| 11 | BRCA2-11OR | 5′-ACC ACT GGC TAT CCT AAA TG-3′ | 20 | | 64 |
| 11 | BRCA2-11PF | 5′-TGA AGA TAT TTG CGT TGA GG-3′ | 20 | 355 | 65 |
| 11 | BRCA2-11PR | 5′-GTC AGC AAA AAC CTT ATG TG-3′ | 20 | | 66 |
| 11 | BRCA2-11QF | 5′-ACG AAA ATT ATG GCA GGT TGT-3′ | 21 | 337 | 67 |
| 11 | BRCA2-11QR | 5′-CTT GTC TTG CGT TTT GTA ATG-3′ | 21 | | 68 |
| 11 | BRCA2-11RF | 5′-GCT TCA TAA GTC AGT CTC AT-3′ | 20 | 360 | 69 |
| 11 | BRCA2-11RR | 5′-TCA AAT TCC TCT AAC ACT CC-3′ | 20 | | 70 |
| 11 | BRCA2-11SF-M13 | 5′-TGT AAA ACG ACG GCC AGT TAC AGC AAG TGG AAA GC-3′ | 35 | 458 | 71 |
| 11 | BRCA2-11SR-M13 | 5′-CAG GAA ACA GCT ATG ACC AAG TTT CAG TTT TAC CAA T-3′ | 37 | | 72 |
| 11 | BRCA2-11TF | 5′-GTT CTT CAG AAA ATA ATC ACT C-3′ | 22 | 344 | 73 |
| 11 | BRCA2-11TR | 5′-TGT AAA AAG AGA ATG TGT GGC-3′ | 21 | | 74 |
| 11 | BRCA2-11UF-M13 | 5′-TGT AAA ACG ACG GCC AGT ACT TTT TCT GAT GTT CCT GTG-3′ | 39 | 328 | 75 |
| 11 | BRCA2-11UR-M13 | 5′-CAG GAA ACA GCT ATG ACC TAA AAA TAG TGA TTG GCA ACA-3′ | 39 | | 76 |
| 12 | BRCA2-12F/M13F | 5′-TGT AAA ACG ACG GCC AGT AGT GGT GTT TTA AAG TGG TCA | 42 | 391 | 77 |
| | | AAA-3′ | | | |
| 12 | BRCA2-12R/M13R | 5′-CAG GAA ACA GCT ATG ACC GGA TCC ACC TGA GGT CAG AAT | 40 | | 78 |
| | | A-3′ | | | |
| 13 | BRCA2/13-2F | 5′-TAA CAT TTA AGC ATC CGT TAC-3′ | 21 | 310 | 79 |
| 13 | BRCA2/13-2R | 5′-AAA CGA GAC TTT TCT CAT ACT GTA TTA G-3′ | 28 | | 80 |
| 14 | BRCA2-14F | 5′-ACC ATG TAG CAA ATG AGG GTC T-3 | 22 | 391 | 81 |
| 14 | BRCA2-14AR | 5′-GCT TTT GTC TGT TTT CCT CCA A-3′ | 22 | | 82 |
| 15 | BRCA2-15-2F | 5′-CCA GGG GTT GTG CTT TTT AAA-3′ | 21 | 284 | 83 |
| 15 | BRCA2-15FUT/M13-R | 5′-CAG GAA ACA GCT ATG ACC ACT CTG TCA TAA AAG CCA TC-3′ | 38 | | 84 |
| 16 | BRCA2-16AF | 5′-TTT GGT TTG TTA TAA TTG TTT TTA-3′ | 24 | 394 | 85 |
| 16 | BRCA2-16AR | 5′-CCA ACT TTT TAG TTC GAG AG-3′ | 20 | | 86 |
| 17 | BRCA2-17F | 5′-TTC AGT ATC ATC CTA TGT G-3′ | 19 | 282 | 87 |
| 17 | BRCA2-17AR | 5′-AGA AAC CTT AAC CCA TAC TG-3′ | 20 | | 88 |
| 18 | BRCA2-18FUT/M13-AF | 5′-TGT AAA ACG ACG GCC AGT GAA TTC TAG AGT CAC ACT TCC-3′ | 39 | 275 | 89 |
| 18 | BRCA2-18R/M13R | 5′-CAG GAA ACA GCT ATG ACC TTT AAC TGA ATC AAT GAC TG-3′ | 38 | | 90 |
| 19 | BRCA2-19F/M13F | 5′-TGT AAA ACG ACG GCC AGT AAG TGA ATA TTT TTA AGG CAG | 41 | 355 | 91 |
| | | TT-3′ | | | |
| 19 | BRCA2-19FUT/M13-R | 5′-CAG GAA ACA GCT ATG ACC AAG AGA CCG AAA CTC CAT CTC-3′ | 39 | | 92 |
| 20 | BRCA2-20F/M13F | 5′-TGT AAA ACG ACG GCC AGT CAC TGT GCC TGG CCT GAT AC-3′ | 38 | 296 | 93 |
| 20 | BRCA2-20R/M13R | 5′-CAG GAA ACA GCT ATG ACC ATG TTA AAT TCA AAG TCT CTA-3′ | 39 | | 94 |
| 21 | BRCA2-21F/M13F | 5′-TGT AAA ACG ACG GCC AGT GGG TGT TTT ATG CTT GGT TCT-3′ | 39 | 304 | 95 |
| 21 | BRCA2-21R/M13R | 5′-CAG GAA ACA GCT ATG ACC CAT TTC AAC ATA TTC CTT CCT | 40 | | 96 |
| | | G-3′ | | | |
| 22 | BRCA2-22F-1A | 5′-AAC CAC ACC CTT AAG ATG A-3′ | 19 | 453 | 97 |
| 22 | BRCA2-22R-1A | 5′-GCA TTA GTA GTG GAT TTT GC-3′ | 20 | | 98 |
| 23 | BRCA2-23FII | 5′-TCA CTT CCA TTG CAT C-3′ | 16 | 290 | 99 |
| 23 | BRCA2-23RII | 5′-TGC CAA CTG GTA GCT CC-3′ | 17 | | 100 |
| 24 | BRCA2-24 2F | 5′-TAC AGT TAG CAG CGA CAA AA-3′ | 20 | 373 | 101 |
| 24 | BRCA2-24R/M13R | 5′-CAG GAA ACA GCT ATG ACC ATT TGC CAA CTG GTA GCT CC-3′ | 38 | | 102 |
| 25 | BRCA2-25F-7/23 | 5′-GCT TTC GCC AAA TTC AGC TA-3′ | 20 | 427 | 103 |
| 25 | BRCA2-25R-7/23 | 5′-TAC CAA AAT GTG TGG TGA TG-3′ | 20 | | 104 |
| 26 | BRCA2/26-2F | 5′-AAT CAC TGA TAC TGG TTT TG-3′ | 20 | 530 | 105 |
| 26 | BRCA2/26-2R | 5′-TAT ACT TAC AGG AGC CAC AT-3′ | 20 | | 106 |
| 27A | BRCA2-27AF-1A | 5′-CTG TGT GTA ATA TTT GCG-3′ | 18 | 495 | 107 |
| 27A | BRCA2-27AR/M13R | 5′-CAG GAA ACA GCT ATG ACG GCA AGT TCT TCG TCA GCT ATT | 40 | | 108 |
| | | G-3′ | | | |
| 27B | BRCA2-27BF/M13F | 5′-TGT AAA ACG ACG GCC AGT GAA TTC TCC TCA GAT GAC TCC | 40 | 417 | 109 |
| | | A-3′ | | | |
| 27B | BRCA2-27BR/M13R | 5′-CAG GAA ACA GCT ATG ACC TCT TTG CTC ATT GTG CAA CA-3′ | 38 | | 110 |

TABLE III
NORMAL PANEL TYPING

| Position | Nucleotide | Amino Acid | | | | | | |
|---|---|---|---|---|---|---|---|---|
| nt/codon | Change | Change | 1 | 2 | 3 | 4 | 5 | Frequency |
| 1093/289 | AAT → CAT | Asn → His | A/A | A/C | A/A | A/A | A/C | A = .8 |
| | | | | | | | | C = .2 |
| 1342/372 | AAT → CAT | Asn → His | A/C | A/A | A/C | A/C | A/C | A = 0.6 |
| | | | | | | | | C = 0.4 |
| 1593/455 | TCA → TCG | Ser → Ser | A/A | A/A | A/A | A/A | A/G | A = 0.9 |
| | | | | | | | | G = 0.1 |
| 2457/743 | CAT → CAC | His → His | T/T | C/T | T/T | T/T | C/T | T = 0.8 |
| | | | | | | | | C = 0.2 |
| 2908/894 | GTA → ATA | Val → Ile | G/G | G/G | G/G | G/G | A/G | G = 0.9 |
| | | | | | | | | A = 0.1 |
| 3199/991 | AAC → GAC | Asn → Asp | A/A | A/G | A/A | A/A | A/G | A = 0.8 |
| | | | | | | | | G = 0.2 |
| 3624/1132 | AAA → AAG | Lys → Lys | A/A | A/G | A/A | A/G | A/A | A = 0.8 |
| | | | | | | | | G = 0.2 |
| 4035/1269 | GTT → GTC | Val → Val | C/T | T/T | T/T | T/T | T/T | T = 0.9 |
| | | | | | | | | C = 0.1 |
| 7470/2414 | TCA → TCG | Ser → Ser | A/A | A/G | A/A | A/G | A/A | A = 0.8 |
| | | | | | | | | G = 0.2 |
| 9079/2951 | GCC → ACC | Ala → Thr | G/G | G/G | G/G | G/G | A/G | G = 0.9 |
| | | | | | | | | A = 0.1 |

### Example 2

### Determination of a Normal Individual Using BRCA2^{(OMI 1-5)} and the Ten Polymorphisms for Reference

A person skilled in the art of genetic susceptibility testing will find the present invention useful for:
a) identifying individuals having a normal BRCA2 gene;
b) avoiding misinterpretation of normal polymorphisms found in the normal population.

Sequencing was carried out as in EXAMPLE 1 using a blood sample from the patient in question. However, the BRCA2 sequences were used for reference and any polymorphic sites seen in the patient were compared to the nucleic acid sequences listed above for normal codons at each polymorphic site. A normal sample is one which is comparable to the BRCA2^{(omi 1-5)} sequences and contains only minor variations which occur at minor polymorphic sites. The allelic variations which occur at each of the polymorphic sites are paired here for reference.
AAT (Asn) and CAT (His) at position 1093 (codon 289)
CAT (His) and AAT (Asn) at position 1342 (codon 372)
TCA (Ser) and TCG (Ser) at position 1593 (codon 455)
CAT (His) and CAC (His) at position 2457 (codon 743)
GTA (Val) and ATA (Ile) at position 2908 (codon 894)
AAC (Asn) and GAC (Asp) at position 3199 (codon 991)
AAA (Lys) and AAG (Lys) at position 3624 (codon 1132)
GTT (Val) and GTC (Val) at position 4035 (codon 1269)
TCA (Ser) and TCG (Ser) at position 7470 (codon 2414)
GCC (Ala) and ACC (Thr) at position 9079 (codon 2951)

The availability of these polymorphic pairs provides added assurance that one skilled in the art can correctly interpret the polymorphic variations without mistaking a normal variation for a mutation.

All exons of the BRCA2 gene are subjected to direct dideoxy sequence analysis by asymmetric amplification using the polymerase chain reaction (PCR) to generate a single stranded product amplified from this DNA sample. Shuldiner, et al., Handbook of Techniques in Endocrine Research, p. 457-486, DePablo, F., Scanes, C., eds., Academic Press, Inc., 1993. Fluorescent dye is attached for automated sequencing using the Taq Dye Terminator Kit (Perkin-Elmer® cat #401628). DNA sequencing is performed in both forward and reverse directions on an Applied Biosystems, Inc. (ABI) automated sequencer (Model 377). The software used for analysis of the resulting data is “Sequence Navigator” purchased through ABI.

### 1. Polymerase Chain Reaction (PCR) Amplification

The PCR primers used to amplify a patient's sample BRCA2 gene are listed in TABLE II. The primers were synthesized on a DNA/RNA Synthesizer Model 394®. Thirty-five cycles are of amplification are performed, each consisting of denaturing (95° C.; 30 seconds), annealing (55° C.; 1 minute), and extension (72° C.; 90 seconds), except during the first cycle in which the denaturing time is increased to 5 minutes and during the last cycle in which the extension time is increased to 5 minutes.

PCR products are purified using Qia-Quick® PCR purification kits (Qiagen®, cat #28104; Chatsworth, Calif.). Yield and purity of the PCR product are determined spectrophotometrically at OD₂₆₀ on a Beckman DU 650 spectrophotometer.

### 2. Dideoxy Sequence Analysis

Fluorescent dye is attached to PCR products for automated sequencing using the Taq Dye Terminator Kit (Perkin-Elmer® cat #401628). DNA sequencing is performed in both forward and reverse directions on an Applied Biosystems, Inc. (ABI) Foster City, Calif., automated sequencer (Model 377). The software used for analysis of the resulting data is “Sequence Navigator®” purchased through ABI. The BRCA2^{(omi 1-5)} sequences were entered sequentially into the Sequence Navigator software as the standards for comparison. The Sequence Navigator software compares the patient sample sequence to each BRCA2^{(omi 1-5)} standard, base by base. The Sequence Navigator highlights all differences between the standards (omi 1-5) and the patient's sample sequence.

A first technologist checks the computerized results by comparing visually the BRCA2^{(omi 1-5)} standards against the patient's sample, and again highlights any differences between the standard and the sample. The first primary technologist then interprets the sequence variations at each position along the sequence. Chromatograms from each sequence variation are generated by the Sequence Navigator and printed on a color printer. The peaks are interpreted by the first primary technologist and a second primary technologist. A secondary technologist then reviews the chromatograms. The results are finally interpreted by a geneticist. In each instance, a variation is compared to known normal polymorphisms for position and base change.

### 3. Results

The patient's BRCA2 sequence was found to be heterozygous at seven nucleotide positions: 1093 (A/C), 1342 (A/C), 1593 (A/G), 2457 (C/T), 2908 (A/G), 3199 (A/G) and 9079 (A/G). In addition, this changes five amino acids in the polypeptide product: Asn to His at codon 289, Asn to His at codon 372, Val to Ile at codon 894, Asn to Asp at codon 991, and Ala to Thr at codon 2951. The question arises whether any or all of these changes have significance to the patient. Comparison of the patient's results to the BRCA (omi 15) haplotypes demonstrates that it matches one of the BRCA2 omi standards (#5), and thus the patient sample is interpreted as carrying a normal gene sequence without causing any elevation in their risk status for breast cancer.

### Example 3

### Determining the Presence of a Mutation in Exon 11 of the BRCA2 Gene using BRCA2^{(omi 1-5)}

A person skilled in the art of genetic susceptibility testing will find the present invention useful for determining the presence of a known or previously unknown mutation in the BRCA2 gene. A list of mutations of BRCA2 is publicly available in the Breast Cancer Information Core at http://www.nchgr.nih.gov/dir/lab_transfer/bic. This data site became publicly available on Nov. 1, 1995. Friend, S. et al. Nature Genetics 11:238, (1995).

In this example, a mutation in exon 11 is characterized by amplifying the region of the mutation with a primer set which amplifies the region of the mutation. Sequencing was carried out as in Example 1 using a blood sample from the patient in question. Specifically, exon 11 of the BRCA2 gene is subjected to direct dideoxy sequence analysis by asymmetric amplification using the polymerase chain reaction (PCR) to generate a single stranded product amplified from this DNA sample. Shuldiner, et al., Handbook of Techniques in Endocrine Research, p. 457-486, DePablo, F., Scanes, C., eds., Academic Press, Inc., 1993. Fluorescent dye is attached for automated sequencing using the Taq Dye Terminator Kit (Perkin-Elmer® cat #401628). DNA sequencing is performed in both forward and reverse directions on an Applied Biosystems, Inc. (ABI) automated sequencer (Model 377). The software used for analysis of the resulting data is “Sequence Navigator” purchased through ABI.

### 1. Polymerase Chain Reaction (PCR) Amplification

Genomic DNA (100 nanograms) extracted from white blood cells of the subject is amplified in a final volume of 25 microliters containing 1 microliter (100 nanograms) genomic DNA, 2.5 microliters 10×PCR buffer (100 mM Tris, pH 8.3, 500 mM KCl, 1.2 mM MgCl₂), 2.5 microliters 10×dNTP mix (2 mM each nucleotide), 2.5 microliters forward primer (BRCA2-11Q-F, 10 micromolar solution), 2.5 microliters reverse primer (BRCA2-11Q-R, 10 micromolar solution), and 1 microliter Taq polymerase (5 units), and 13 microliters of water.

The PCR primers used to amplify segment Q of exon 11 (where the mutation 6174delT is found) are as follows:

BRCA2-11Q-F:
5′- ACG′ AAA′ ATT′ ATG′ GCA′ GGT′ TGT-3′
BRCA2-11Q-R:
5′- CTT′ GTC′ TTG′ CGT′ TTT′ GTA′ ATG-3′

The primers are synthesized on an DNA/RNA Synthesizer Model 394®. Thirty-five cycles are performed, each consisting of denaturing (95° C.; 30 seconds), annealing (55° C.; 1 minute), and extension (72° C.; 90 seconds), except during the first cycle in which the denaturing time is increased to 5 minutes, and during the last cycle in which the extension time is increased to 5 minutes.

PCR products are purified using Qia-Quick® PCR purification kits (Qiagen®, cat #28104; Chatsworth, Calif.). Yield and purity of the PCR product are determined spectrophotometrically at OD₂₆₀ on a Beckman DU 650 spectrophotometer.

### 2. Dideoxy Sequence Analysis

Fluorescent dye is attached to PCR products for automated sequencing using the Taq Dye Terminator Kit (Perkin-Elmer® cat #401628). DNA sequencing is performed in both forward and reverse directions on an Applied Biosystems, Inc. (ABI) Foster City, Calif., automated sequencer (Model 377). The software used for analysis of the resulting data is “Sequence Navigator®” purchased through ABI. The BRCA2^{(omi 1-5)} sequence is entered into the Sequence Navigator software as the Standard for comparison. The Sequence Navigator software compares the sample sequence to the BRCA2^{(omi)} standard, base by base. The Sequence Navigator highlights all differences between the BRCA2^{(omi)} normal DNA sequence and the patient's sample sequence.

A first technologist checks the computerized results by comparing visually the BRCA2^{(omi 1-5)} standard against the patient's sample, and again highlights any differences between the standard and the sample. The first primary technologist then interprets the sequence variations at each position along the sequence. Chromatograms from each sequence variation are generated by the Sequence Navigator and printed on a color printer. The peaks are interpreted by the first primary technologist and a second primary technologist. A secondary technologist then reviews the chromatograms. The results are finally interpreted by a geneticist. In each instance, a sequence variation is compared to known normal polymorphisms for position and base change. The ten frequent polymorphisms which occur in BRCA2 are:
AAT (Asn) and CAT (His) at position 1093 (codon 289)
CAT (His) and AAT (Asn) at position 1342 (codon 372)
TCA (Ser) and TCG (Ser) at position 1593 (codon 455)
CAT (His) and CAC (His) at position 2457 (codon 743)
GTA (Val) and ATA (Ile) at position 2908 (codon 894)
AAC (Asn) and GAC (Asp) at position 3199 (codon 991)
AAA (Lys) and AAG (Lys) at position 3624 (codon 1132)
GTT (Val) and GTC (Val) at position 4035 (codon 1269)
TCA (Ser) and TCG (Ser) at position 7470 (codon 2414)
GCC (Ala) and ACC (Thr) at position 9079 (codon 2951)

### 3. Results

Using the above PCR amplification and standard fluorescent sequencing technology, the 6174delT mutation may be found. Mutations are noted by the length of non-matching sequence variation. Such a lengthy mismatch pattern occurs with deletions and insertions. This mutation is named in accordance with the suggested nomenclature for naming mutations, Beaudet, A et al., Human Mutation 2:245-248, (1993). The 6174delT mutation at codon 1982 of the BRCA2 gene lies in segment “Q” of exon 11. The DNA sequence results demonstrate the presence of a one base pair deletion of a T at nucleotide 6174 of the BRCA2^{(omi 1-5)} sequences. This mutation interrupts the normal reading frame of the BRCA2 transcript, resulting in the appearance of an in-frame terminator (TAG) at codon position 2003. This mutation is, therefore, predicted to result in a truncated, and most likely, non-functional protein.

### Example 4

### Generation of Monoclonal and Polyclonal Antibodies Using GST-BRCA2 Fusion Protein as an Immunogen

DNA primers are used to amplify a fragment of BRCA2 using PCR technology. The product is then digested with suitable restriction enzymes and fused in frame with the gene encoding glutathione S-transferase (GST) in Escherichia coli using GST expression vector pGEX (Pharmacia Biotech Inc.) The expression of the fusion protein is induced by the addition of isopropyl-β-thiogalactopyranoside. The bacteria are then lysed and the overexpressed fusion protein is purified with glutathione-sepharose beads. The fusion protein is then verified by SDS/PAGE gel and N-terminus protein sequencing. The purified protein is used to immunize rabbits according to standard procedures described in Harlow & Lane (1988). Polycolonal antibody is collected from the serum several weeks after and purified using known methods in the art. Monoclonal antibodies against all or fragments of BRCA2 protein, polypeptides, or functional equivalents are obtained using hybridoma technology, see also Harlow & Lane (1988). The BRCA2 protein or polypeptide is coupled to the carrier keyhole limpet hemocyanin in the presence of glutaraldehyde. The conjugated immunogen is mixed with an adjuvant and injected into rabbits. Spleens from antibody-containing rabbits are removed. The B-cells isolated from spleen are fused to myeloma cells using polyethylene glycol (PEG) to promote fusion. The hybrids between the myeloma and B-cells are selected and screened for the production of antibodies to immunogen BRCA2 protein or polypeptide. Positive cells are recloned to generate monoclonal antibodies.

### Example 5

### Detection of BRCA2 Expression in Human Tissues and Cell Lines

The expression of BRCA2 in human tissues is determined using Northern blot analysis. Human tissues include those from pancreas, testis, prostate, ovary, breast, small intestine, and colon are obtained from Clontech Laboratories, Inc., Palo Alto, Calif. The poly(A)+ mRNA Northern blots from different human tissues is hybridized to BRCA2 cDNA probes according to manufacture protocol. The expression level is further conformed by RT-PCR using oligo-d(T) as a primer and other suitable primers.

For Northern Blot analysis of cancer cell lines, the human ovarian cancer cell line SKOV-3 and the human breast cancer cell line MCF-7 are obtained from the American Type Culture Collection. Total RNA is prepared by lysing cell in the presence of guanidinium isocyanate. Poly(A)⁺ mRNA is isolated using the PolyATract mRNA isolation system from Promega, Madison, Wis. The isolated RNA is then electrophoresed under denaturing conditions and transferred to Nylon membrane. The probe used for Northern blot is a fragment of BRCA2 sequence obtained by PCR amplification. The probes are labeled with [α-³²P] dCTP using a random-primed labeling kit (Amersham Life Science, Arlington Heights, Ill.).

### Example 6

### Expression of the BRCA2 Protein

The whole-cell extracts of BRCA2 transfected cells are subjected to immunoprecipitation and immunoblotting to determine the BRCA2 protein level. The BRCA2 protein or polypeptide is immunoprecipitated using anti-BRCA2 antibodies prepared according to Example 4. Samples are then fractionated using SDS/PAGE gel and transferred to nitrocellulose. Western blot of the BRCA2 protein or polypeptide is performed with the indicated antibodies. Antibody reaction is revealed using enhanced chemiluminescence reagents (Dupont New England Nuclear, Boston, Mass.).

### Example 7

### Use of the BRCA2^{(omi 1-5)} Gene Therapy

The growth of ovarian or breast cancer may be arrested by increasing the expression of the BRCA2 gene where inadequate expression of that gene is responsible for hereditary ovarian or breast cancer. Gene therapy may be performed on a patient to reduce the size of a tumor. The LXSN vector may be transformed with a BRCA2^{(omi 1-5)} coding sequence as presented SEQ ID NO:4, 6, 8, 10, or 12 or a fragment thereof.

### Vector

The LXSN vector is transformed with a fragment of the wildtype BRCA2^{(omi 1-5)} coding sequence as set forth in SEQ ID NO:4, 6, 8, 10, or 12. The LXSN-BRCA2^{(omi 1-5)} retroviral expression vector is constructed by cloning a Sal I linkered BRCA2^{(omi 1-5)} cDNA or fragments thereof into the Xho I site of the vector LXSN. Constructs are confirmed by DNA sequencing. See Holt et al., Nature Genetics 12: 298-302 (1996). Retroviral vectors are manufactured from viral producer cells using serum free and phenol-red free conditions and tested for sterility, absence of specific pathogens, and absence of replication-competent retrovirus by standard assays. Retrovirus is stored frozen in aliquots which have been tested.

Patients receive a complete physical exam, blood, and urine tests to determine overall health. They may also have a chest X-ray, electrocardiogram, and appropriate radiologic procedures to assess tumor stage.

Patients with metastatic ovarian cancer are treated with retroviral gene therapy by infusion of recombinant LXSN-BRCA2^{(omi 1-5)} retroviral vectors into peritoneal sites containing tumor, between 10⁹ and 10¹⁰ viral particles per dose. Blood samples are drawn each day and tested for the presence of retroviral vector by sensitive polymerase chain reaction (PCR)-based assays. The fluid which is removed is analyzed to determine:

1. The percentage of cancer cells which are taking up the recombinant LXSN-BRCA2^{(omi 1-5)} retroviral vector combination. Successful transfer of BRCA1 gene into cancer cells has been shown by both RT-PCR analysis and in situ hybridization. RT-PCR is performed with by the method of Thompson et al., Nature Genetics 9: 444-450 (1995), using primers derived from a BRCA2^{(omi 1-5)} coding sequence as in SEQ ID NO:4, 6, 8, 10, or 12 or fragments thereof. Cell lysates are prepared and immunoblotting is performed by the method of Jensen et al., Nature Genetics 12: 303-308 (1996) and Jensen et al., Biochemistry 31: 10887-10892 (1992).

2. Presence of programmed cell death using APOTAG® in situ apoptosis detection kit (ONCOR, INC., Gaithersburg, Md.) and DNA analysis.

3. Measurement of BRCA2 gene expression by slide immunofluorescence or Western blot.

Patients with measurable disease are also evaluated for a clinical response to LXSN-BRCA2^{(omi 1-5)} especially those that do not undergo a palliative intervention immediately after retroviral vector therapy. Fluid cytology, abdominal girth, CT scans of the abdomen, and local symptoms are followed.

For other sites of disease, conventional response criteria are used as follows:
1. Complete Response (CR), complete disappearance of all measurable lesions and of all signs and symptoms of disease for at least 4 weeks.
2. Partial Response (PR), decrease of at least 50% of the sum of the products of the 2 largest perpendicular diameters of all measurable lesions as determined by 2 observations not less than 4 weeks apart. To be considered a PR, no new lesions should have appeared during this period and none should have increased in size.
3. Stable Disease, less than 25% change in tumor volume from previous evaluations.
4. Progressive Disease, greater than 25% increase in tumor measurements from prior evaluations. The number of doses depends upon the response to treatment.

### Example 8

### Protein Replacement Therapy

Therapeutically elevated level of functional BRCA2 protein may alleviate the absence or reduced endogenous BRCA2 tumor suppressing activity. Breast or ovarian cancer is treated by the administration of a therapeutically effective amount of the BRCA2 protein, a polypeptide, or its functional equivalent in a pharmaceutically acceptable carrier. Clinically effective delivery method is applied either locally at the site of the tumor or systemically to reach other metastasized locations with known protocols in the art. These protocols may employ the methods of direct injection into a tumor or diffusion using time release capsule. A therapeutically effective dosage is determined by one of skill in the art.

Breast or ovarian cancer may be prevented by the administration of a prophylactically effective amount of the BRCA2 protein, polypeptide, or its functional equivalent in a pharmaceutically acceptable carrier. Individuals with known risk for breast or ovarian cancer are subjected to protein replacement therapy to prevent tumorigenesis or to decrease the risk of cancer. Elevated risk for breast and ovarian cancer includes factors such as carriers of one or more known BRCA1 and BRCA2 mutations, late child bearing, early onset of menstrual period, late occurrence of menopause, and certain high risk dietary habits. Clinically effective delivery method is used with known protocols in the art, such as administration into peritoneal cavity, or using an implantable time release capsule. A prophylactically effective dosage is determined by one of skill in the art.

### TABLE OF REFERENCES

1. Sanger, F., et al., J. Mol. Biol. 42:1617, (1980).
2. Beaucage, et al., Tetrahedron Letters 22:1859-1862, (1981).
3. Maniatis, et al. in Molecular Cloning: A Laboratory Manual, Cold Spring Harbor, N.Y., p 280-281, (1982).
4. Conner, et al., Proc. Natl. Acad. Sci. U.S.A. 80:278, (1983)
5. Saiki, et.al., Bio/Technology 3:1008-1012, (1985)
6. Landgren, et al., Science 241:1007,(1988)
7. Landgren, et al., Science 242 :229-237, (1988).
8. PCR. A Practical Approach, ILR Press, Eds. M. J. McPherson, P. Quirke, and G. R. Taylor, (1992).
9. Easton et al., American Journal of Human Genetics 52:678-701, (1993).
10. U.S. Pat. No. 4,458,066.
11. Rowell, S., et al., American Journal of Human Genetics 55:861-865, (1994)
12. Miki, Y. et al., Science 266:66-71, (1994).
13. Wooster, R. et al., Science 265:2088-2090, (1994).
14. Wooster, R. et al., Nature 378:789-792, (1995).
15. Beaudet, A et al., Human Mutation 2:245-248, (1993).
16. Friend, S. et al. Nature Genetics 11:238, (1995).
17. Teng et al, Nature Genetics 13: 241-244 (1996).
18. Couch et al, Nature Genetics 13: 123-125 (1996).
19. Tartigan et al, Nature Genetics 12: 333-337 (1996).
20. Phelan et al, Nature Genetics 13: 120-122 (1996).
21. Schubert et al, American Journal of Human Genetics 60: 1031-1040 (1996).
22. Sambrook, et al., Molecular Cloning: A Laboratory Manual, Second Edition, Cold Spring Harbor Laboratory, Cold Spring Harbor, N.Y. (1989).
23. Bertwistle and Ashworth, Curr. Opin. Genet. Dev. 8(1): 14-20 (1998).
24. Zhang et al., Cell 92:433-436 (1998).
25. Sharan et al., Nature 386:804-810 (1997).
26. Katagiri et al., Genes, Chromosomes & Cancer 21:217-222 (1988).
27. Crooke, Annu. Rev. Pharmacol. Toxicol. 32:329-376 (1992)
28. Robinson-Benion and Holt, Methods Enzymol. 254:363-375 (1995).
29. Harlow & Lane, Antibodies: A Laboratory Manual, Cold Spring Harbor Laboratory, Cold Spring Harbor, N.Y., 1988.
30. Shuldiner, et al., Handbook of Techniques in Endocrine Research, p. 457-486, DePablo, F., Scanes, C., eds., Academic Press,. Inc., 1993.
31. Holt et al., Nature Genetics 12: 298-302 (1996).
32. Thompson et al., Nature Genetics 9: 444-450 (1995).
33. Jensen et al., Nature Genetics 12: 303-308 (1996)
34. Jensen et al., Biochemistry 31: 10887-10892 (1992).

Although the invention has been described with reference to the presently preferred embodiments, it should be understood that various modifications can be made without departing from the spirit of the invention. Accordingly, the invention is limited only by the following claims.

1-60. (canceled)

61. An isolated nucleic acid comprising:
(a) 18 contiguous nucleotides spanning the 3′ exon/intron junction of exon 15 according to FIG. 2D;
(b) 18 contiguous nucleotides spanning the 5′ exon/intron junction of exon 16 according to FIG. 2D;
(c) the nucleotide
sequence 5′-GTGTTCTCATAAACAGgtatgtgt-3′;
(d) the nucleotide
sequence 5′-tttttcttttttgtgtgtgtttattttgtgtagCTGTA
TACGTATGGCGTTTC-3′;
(e) the nucleotide sequence of exon 15 and having the nucleotide
sequence 5′- GTGTTCTCATAAACAGgtatgtgt-3′;
(f) the nucleotide sequence of SEQ ID NO:2; or
(g) the nucleotide sequence of SEQ ID NO:3.

62. A method of amplifying exon 15 of a BRCA2 gene,
wherein the sequence of the last 16 nucleotides of exon 15 is 5′-GTGTTCTCATAAACAG-3′, the method comprising:
obtaining a sample containing genomic DNA from a human subject;
amplifying exon 15 or two or more portions thereof from said genomic DNA.

63. The method of claim 62, wherein said amplifying step comprises PCR to obtain (1) a first isolated nucleic acid comprising the 5′ intron/exon junction of exon 15 according to SEQ ID NO:2 and (2) a second isolated nucleic acid comprising the 3′ exon/intron junction of exon 15 according to SEQ ID NO:2.

64. A method of amplifying exon 16 of a BRCA2 gene, wherein the sequence of the first 20 nucleotides of exon 16 is 5′-CTGTATACGTATGGCGTTTC-3′, the method comprising:
obtaining a sample containing genomic DNA from a human subject;
amplifying exon 16 or two or more portions thereof from said genomic DNA.

65. The method of claim 64, wherein said amplifying step comprises PCR to obtain (1) a first isolated nucleic acid comprising the 5′ intron/exon junction of exon 16 according to SEQ ID NO:3 and (2) a second isolated nucleic acid comprising the 3′ exon/intron junction of exon 16 according to SEQ ID NO:3.
